SEQUENCE LISTING (60 sequences)

SEQ ID NO: 1 - DNA, 34 bp, Artificial Sequence
  Description of Artificial Sequence Synthetic primer
gtcacgtgtaaaatgggcatgaacgcccttcgca

SEQ ID NO: 2 - DNA, 39 bp, Artificial Sequence
  Description of Artificial Sequence Synthetic primer
gcagtcatatttgcaagtgtacctggcaggaaaggacat

SEQ ID NO: 3 - DNA, 36 bp, Artificial Sequence
  Description of Artificial Sequence Synthetic primer
ggattgaagtcctgggggttggagaagaaactgggg

SEQ ID NO: 4 - DNA, 36 bp, Artificial Sequence
  Description of Artificial Sequence Synthetic primer
aggcggagccatatcatccaccccattttgcctatt

SEQ ID NO: 5 - DNA, 31 bp, Artificial Sequence
  Description of Artificial Sequence Synthetic primer
gtcatctgcctgccccactcccagactgatt

SEQ ID NO: 6 - DNA, 37 bp, Artificial Sequence
  Description of Artificial Sequence Synthetic primer
caggatggatgtccaataccctgtctccaaggacacc

SEQ ID NO: 7 - DNA, 36 bp, Artificial Sequence
  Description of Artificial Sequence Synthetic primer
acatccatcctgggttctggtgcaactgtccagttg

SEQ ID NO: 8 - DNA, 32 bp, Artificial Sequence
  Description of Artificial Sequence Synthetic primer
ccgccgccccctggcctgtctccattcccgcg

SEQ ID NO: 9 - DNA, 38 bp, Artificial Sequence
  Description of Artificial Sequence Synthetic primer
ggagctggacatcccaagatcctgtctttctgatgctg

SEQ ID NO: 10 - DNA, 36 bp, Artificial Sequence
  Description of Artificial Sequence Synthetic primer
gaagacccctaaatgcacagccacactttgtcttac

SEQ ID NO: 11 - DNA, 28 bp, Artificial Sequence
  Description of Artificial Sequence Synthetic primer
gtcacgtgtaaaatgggcatgaacgccc

SEQ ID NO: 12 - DNA, 33 bp, Artificial Sequence
  Description of Artificial Sequence Synthetic primer
gcagtcatatttgcaagtgtacctggcaggaaa

SEQ ID NO: 13 - DNA, 30 bp, Artificial Sequence
  Description of Artificial Sequence Synthetic primer
ggattgaagtcctgggggttggagaagaaa

SEQ ID NO: 14 - DNA, 30 bp, Artificial Sequence
  Description of Artificial Sequence Synthetic primer
aggcggagccatatcatccaccccattttg

SEQ ID NO: 15 - DNA, 25 bp, Artificial Sequence
  Description of Artificial Sequence Synthetic primer
gtcatctgcctgccccactcccaga

SEQ ID NO: 16 - DNA, 31 bp, Artificial Sequence
  Description of Artificial Sequence Synthetic primer
caggatggatgtccaataccctgtctccaag

SEQ ID NO: 17 - DNA, 30 bp, Artificial Sequence
  Description of Artificial Sequence Synthetic primer
acatccatcctgggttctggtgcaactgtc

SEQ ID NO: 18 - DNA, 26 bp, Artificial Sequence
  Description of Artificial Sequence Synthetic primer
ccgccgccccctggcctgtctccatt

SEQ ID NO: 19 - DNA, 32 bp, Artificial Sequence
  Description of Artificial Sequence Synthetic primer
ggagctggacatcccaagatcctgtctttctg

SEQ ID NO: 20 - DNA, 30 bp, Artificial Sequence
  Description of Artificial Sequence Synthetic primer
gaagacccctaaatgcacagccacactttg

SEQ ID NO: 21 - DNA, 22 bp, Artificial Sequence
  Description of Artificial Sequence Synthetic primer
tgtaaaatgggcatgaacgccc

SEQ ID NO: 22 - DNA, 27 bp, Artificial Sequence
  Description of Artificial Sequence Synthetic primer
atatttgcaagtgtacctggcaggaaa

SEQ ID NO: 23 - DNA, 24 bp, Artificial Sequence
  Description of Artificial Sequence Synthetic primer
aagtcctgggggttggagaagaaa

SEQ ID NO: 24 - DNA, 24 bp, Artificial Sequence
  Description of Artificial Sequence Synthetic primer
agccatatcatccaccccattttg

SEQ ID NO: 25 - DNA, 19 bp, Artificial Sequence
  Description of Artificial Sequence Synthetic primer
tgcctgccccactcccaga

SEQ ID NO: 26 - DNA, 25 bp, Artificial Sequence
  Description of Artificial Sequence Synthetic primer
ggatgtccaataccctgtctccaag

SEQ ID NO: 27 - DNA, 24 bp, Artificial Sequence
  Description of Artificial Sequence Synthetic primer
atcctgggttctggtgcaactgtc

SEQ ID NO: 28 - DNA, 20 bp, Artificial Sequence
  Description of Artificial Sequence Synthetic primer
ccccctggcctgtctccatt

SEQ ID NO: 29 - DNA, 26 bp, Artificial Sequence
  Description of Artificial Sequence Synthetic primer
ggacatcccaagatcctgtctttctg

SEQ ID NO: 30 - DNA, 24 bp, Artificial Sequence
  Description of Artificial Sequence Synthetic primer
ccctaaatgcacagccacactttg

SEQ ID NO: 31 - DNA, 25 bp, Artificial Sequence
  Description of Artificial Sequence Synthetic primer
ctttggattcctctcccttggaatg

SEQ ID NO: 32 - DNA, 24 bp, Artificial Sequence
  Description of Artificial Sequence Synthetic primer
gggacaccttcatgatggagtcac

SEQ ID NO: 33 - DNA, 27 bp, Artificial Sequence
  Description of Artificial Sequence Synthetic primer
cagtgtatcctgatgtgcagactcgag

SEQ ID NO: 34 - DNA, 33 bp, Artificial Sequence
  Description of Artificial Sequence Synthetic primer
cagtcagtcagtttattggcaagtttccttggc

SEQ ID NO: 35 - DNA, 23 bp, Artificial Sequence
  Description of Artificial Sequence Synthetic primer
gttgggctcagatctgtgataga

SEQ ID NO: 36 - DNA, 24 bp, Artificial Sequence
  Description of Artificial Sequence Synthetic primer
caagggcaatgagatcttaagtaa

SEQ ID NO: 37 - DNA, 22 bp, Artificial Sequence
  Description of Artificial Sequence Synthetic primer
tgtaaaatgggcatgaacgccc

SEQ ID NO: 38 - DNA, 24 bp, Artificial Sequence
  Description of Artificial Sequence Synthetic primer
aagtcctgggggttggagaagaaa

SEQ ID NO: 39 - DNA, 24 bp, Artificial Sequence
  Description of Artificial Sequence Synthetic primer
atcctgggttctggtgcaactgtc

SEQ ID NO: 40 - DNA, 24 bp, Artificial Sequence
  Description of Artificial Sequence Synthetic primer
ccctaaatgcacagccacactttg

SEQ ID NO: 41 - DNA, 20 bp, Artificial Sequence
  Description of Artificial Sequence Synthetic primer
cgtgtcccccaaacacgtgg

SEQ ID NO: 42 - DNA, 16 bp, Artificial Sequence
  Description of Artificial Sequence Synthetic primer
accgccagtgccccgg

SEQ ID NO: 43 - DNA, 17 bp, Artificial Sequence
  Description of Artificial Sequence Synthetic primer
gcgcgccaagcagctcc

SEQ ID NO: 44 - DNA, 28 bp, Artificial Sequence
  Description of Artificial Sequence Synthetic primer
ctcaagtcctcccagtcacctaaggaca

SEQ ID NO: 45 - DNA, 24 bp, Artificial Sequence
  Description of Artificial Sequence Synthetic primer
ctccctaccagggcaccgaagtgt

SEQ ID NO: 46 - DNA, 25 bp, Artificial Sequence
  Description of Artificial Sequence Synthetic primer
gggcaggaagctcatggtgtagttt

SEQ ID NO: 47 - DNA, 20 bp, Artificial Sequence
  Description of Artificial Sequence Synthetic primer
ggctggggtggtttgccttt

SEQ ID NO: 48 - DNA, 23 bp, Artificial Sequence
  Description of Artificial Sequence Synthetic primer
tggcaggtgtttcatcaccgaag

SEQ ID NO: 49 - DNA, 26 bp, Artificial Sequence
  Description of Artificial Sequence Synthetic primer
tccacgagatccaaagatttggagac

SEQ ID NO: 50 - DNA, 26 bp, Artificial Sequence
  Description of Artificial Sequence Synthetic primer
tggggaccgctttgactataaggaca

SEQ ID NO: 51 - DNA, 20 bp, Artificial Sequence
  Description of Artificial Sequence Synthetic primer
ttttaccgccagtgccccgg

SEQ ID NO: 52 - DNA, 30 bp, Artificial Sequence
  Description of Artificial Sequence Synthetic primer
tgactgactgactgcgcgccaagcagctcc

SEQ ID NO: 53 - DNA, 36 bp, Artificial Sequence
  Description of Artificial Sequence Synthetic primer
ctgactgactcaagtcctcccagtcacctaaggaca

SEQ ID NO: 54 - DNA, 32 bp, Artificial Sequence
  Description of Artificial Sequence Synthetic primer
ctgactgactccctaccagggcaccgaagtgt

SEQ ID NO: 55 - DNA, 38 bp, Artificial Sequence
  Description of Artificial Sequence Synthetic primer
tgactgactgactgggcaggaagctcatggtgtagttt

SEQ ID NO: 56 - DNA, 41 bp, Artificial Sequence
  Description of Artificial Sequence Synthetic primer
tgactgactgactgactgactggctggggtggtttgccttt

SEQ ID NO: 57 - DNA, 47 bp, Artificial Sequence
  Description of Artificial Sequence Synthetic primer
gactgactgactgactgactgacttggcaggtgtttcatcaccgaag

SEQ ID NO: 58 - DNA, 53 bp, Artificial Sequence
  Description of Artificial Sequence Synthetic primer
actgactgactgactgactgactgacttccacgagatccaaagatttggagac

SEQ ID NO: 59 - DNA, 55 bp, Artificial Sequence
  Description of Artificial Sequence Synthetic primer
ctgactgactgactgactgactgactgactggggaccgctttgactataaggaca

SEQ ID NO: 60 - DNA, 10778 bp, Homo sapiens
cccttacgccctcctgaagcaggacactcttatgccctcaatgacacctgagacaacacacagagccctcttggcaccaaacatgacttcaatattctcctggtgtcaagttcagtgccaacttgtttcccatcctcgcatacacctgggggcagtcccaaagccaacacgatgcctttccctccctggccatacctgatgctgatatcagaacccaccaatgtcatcagtgtgtccactgctgatgccagtcccagcactcacctcctgctaaggctggcctgcacttgactcccagggtgatctaggccagtcaatgaaggggggtgaggagaaagttcacatcccagctccctcacttactccctgtgtgacctcactcaagaggatttatggagtctagatctgctcatcttcaaaatgaggatgatcatgatgaaccaacctcattgttacaaagattcaaccagatcatacacacctagacttaatcttcccgtatacgaccatgctcaccaaagggtaactgtcctgtttactaaggaagaatttccctgagggaaggacaggagggctactcatcccttctccactcacacccaccccaggatctgcctctgtgcctaatactggagtttaccccaatctcttctgccactgttccctctgcctgctaatagtagtagcccctgacaaagcaggaatcattcttaaaggagacttaactcaccctcgaatgtgatcttctcttcccaaacactccctttccactggcaggaaaaccaaatccagaaaggggaactaagtacacagagcaggagagatgggagttcagggccactcacacatgtcccctgcccactgtctgttttctgtcctctgtagatctttatataaaatgagaaacataaacaacaatcataatattaatagggatgatacaatcaatctagtgggtttccctgaggatctgggttggaaaccagcggacaacccttgggacacttgagatttttccaccatttgggggttgttattactatttctccagacctagccaatccctctgccaaccgctaactccaggtactcttctccaggtgtggggaaagttcccctgaaatatggctctggtcttcctccccttcccaatcagagatgggcagtggaggttctatggcacccatcctggcctcactctgaggttccaatgaggattctgggcatcaagagacagctctgggcaaaagcaaaatcaagtcagcccctggacccagtgctgggctgctgggctttctgggagaacccgctgggcttgctacacactcctcctcccagaaactccacacccacagccctgggtcttcctagccccgagactttcaagtccatatgcctggaatcccccttcctgagacccttaaccctgcatcctccacaacagaagacccctaaatgcacagccacactttgtcttaccctaataaaacccagacctttggattcctctcccctggaacccccagatccacaactttggggtgcattctcactctcagaccccaaatccaaagcccaagtgctcccctatgcaaatattccaaactcctcagttctacagcttatctgttgccccctcctaaatccacagccctgcggcacccctcctgaagtaccacagatttagtctggaggccccctctctgttcagctgccctggggtccccttatcctcccttgctggctgtgtcccaagctaggcaggattcatggtggggcatgtagttgggaggtgaaatgaggtaattatgtaatcagccaaagtccatccctctttttcaggcagtataaaggcaaaccaccccagccgtcaccatctatcatcccactaccaccatgctggcctcagggatgcttctggtggccttgctggtctgcctgactgtgatggtcttgatgtctgtttggcagcagaggaagagcaaggggaagctgcctccgggacccaccccattgcccttcattggaaactacctgcagctgaacacagagcagatgtacaactccctcatgaaggtgtcccaaggcagggagatgggtggcacggggtgggggctgcctagttggctggggctttgtggcagggggttgaccagtgtggaccagagtcttaggaaatggagttttggagtttcagcatcagaaagacaggatcttgggatgtccagctccctgactgtgagaacctgggtgcgaagcatcccagcacatgacatctcggtgctgggccccattcagagtggaggcttctccctctaaccactcccacccacctccatcagatcagtgagcgctatggccccgtgttcaccattcacttggggccccggcgggtcgtggtgctgtgtggacatgatgccgtcagggaggctctggtggaccaggctgaggagttcagcgggcgaggcgagcaagccaccttcgactgggtcttcaaaggctatggtgagggggtgcccaagagggggaaggtggccaggtggacacgaaggtctcagtgttcccagccttctccctgactctcctgccaactggaggctaaggcagagtccccagtctggtcttccctccccatctcccttcattgtggcctctccatgtgtatccctcacctgtctccagcgtccctgtcctgattcctccctgcctctctctgccccacctccttattctctctcactggagtctcctctttcccctctctctccatctctaaggacatcctgggtttctgttttccagccctggttctctgtcttcatttgtctttttgtccctcttagcttctgggcttctctgtgtttctcatctctccgcatccctttctcacttcttcctctgtcttaggatttcagggtattcctacttccacatcttcagcctccatctcctggtaacagtctctcttccttccagaccctctctgtttctatctcaatatttttctctcttctccagctcagcttaagaatgtttcaccatttttatttcctcctcccagatctccccatatctcacttcccctccctccatcctcttcctccatcactctctttctctccccacttccttcccttcctccatggagtgtccccttatccctctgtttctatgtgcatctctctgtctggcctttctgtttcttttctgattgtcttattctttaaacctggtctctctctctctctctctctctctctctctctctctctctctcctctctctcttctctctctctctcctctctctcttctctctctctctctctctctctcctctctctcttctctctctctctctctctctctctctctctctctctacctcgacatcgtgtttctctgactgagtttgcagctctgctgggcaatggcgctgaatccatctctctcccaccctactccctctctcctccacccttggggagccccttggagctgctccgctcctgcccccgccgccccctggcctgtctccattcccgcgttcacctccccaggcgtggtattcagcaacggggagcgcgccaagcagctccggcgcttctccatcgccaccctgcgggacttcggggtgggcaagcgaggcatcgaggagcgcatccaggaggaggcgggcttcctcatcgacgccctccggggcactggcggtgagcaggggaccccgagtgcgggggcaggagaaggaaaacacccaggacgaggaacccgcgcgcgttctgcctggggatggggactaggtggggaaaggcgcccgcacttccagccctggagtctggcgctgggaatttggctcaacaaggccctgcctcctggaattctgactctcctcagacctctgagttgactctctccccaacccccttctcccgccatacccgcaggcgccaatatcgatcccaccttcttcctgagccgcacagtctccaatgtcatcagctccattgtctttggggaccgctttgactataaggacaaagagttcctgtcactgttgcgcatgatgctaggaatcttccagttcacgtcaacctccacggggcaggtaactggctgcagcccggcccgtgacgcccctaccacaacctgccaactgctcccctacctggagacaggtgccccaaactcccaccgccctccagacagtgtcccctcaaaatcagtccccgatattggacaactggacagttgcaccagaacccaggatggatgtccaataccctgtctccaaggacacctggatagctcaacagatgctccccaaaacagagcctgctggcaggatgcataccctcagctcagctctctcacctgggcacatgttcccatccccaacttaccgtaatttctaacagatgctccctacccagttcttctgaatattttaacacctggacaagtgactgcgtcaaccggcctcctgcatacctgaacacctggtgctgcaaaatccagcccatcataatcatttcacatctacacaaatgtcccagattagcccactggaatatctggccaagcgccctctacttcacccacttaaatacctgaaaacatggacagctgccctaaccaacaccttaaacacataaatatctagacagattgttccctgacacacaaataagtgttccccaaccctttccaatcacacaccttacagaggtgctcccagtgcatcccacttggataggtaaacacctcaacaggtatcccctccacttcaacatcttcaccagccccactttaatacctgagcacctgaacaaaagcccccaatccagacccagtaagtatctggacagctgtctccaaccaagcctacttgaatgcctaaatacctagacaggtgacactcacctcataccagccccacctgaagagctaaacacctggacaggtgtcttccaactcaacttcacttgaatatctgaacacctagatgtgtgctccaatccagcctcgtttaaatacctgaaacctggatatatgtctcagttcttctcacctaaattactagaccgtggccctggtacctaaccttcctgaaaacttagatataagttcctatccggccccactgaaatacctaaacaacgagacagatgcctttaactcagttccttccttgctatgaaacaaatcccattcccatcagctcctgccccgtgacagctgtccttcccttcccatcctctctctgcaaccccagctctatgagatgttctcttcggtgatgaaacacctgccaggaccacagcaacaggcctttcagttgctgcaagggctggaggacttcatagccaagaaggtggagcacaaccagcgcacgctggatcccaattccccacgggacttcattgactcctttctcatccgcatgcaggaggtacaccccagcagccactgcggggagatgcaaagccaggcagagggaaatcagtctgggagtggggcaggcagatgacacaggcccattcaaattaaccctcatcataataatcctcacaattggctgggtgccgtggctaacagcctgtaatcccagcactttgggaggccgaggcaggtggatcacctgaggtcaggagttcgagaccagcctggccaacatggtcaaaccccgtctctactaaaaatccaaaaattagttgggcatggtggcgcgaaggggggcagaggttgcaatgagccaagatcacggcattgcactccagtctgggtgacagaatgaggccctgtgtcaaaaaaaattaattaattgtttaaaaagtaagtgagcctgcatggtcatgcgcatgtgcagttccagctactcaggaggctgaggctggaggattgcttgagctcaggagttggagtccggcctgtgcaacttagcaagaccaagtcagtataagaaaaaaaaaaacaaaaaaaaactgacagctaagttgataattgaaggacagatggtcagcaaggtaaagaaggtgagaaggaagagcattttgggcaaagccagcagccagggcaagggctggaacctagagcgagtctggtagatctagggtccctctttccacctttggtctgaaccaaagagaggtagatccaaaggaaaagccctagaagggccctgagggcaagaggagtgaggttggcctaaagccccctctccctgcaggaggagaagaaccccaacacggagttctacttgaaaaacctggtgatgaccacgttgaacctcttcattgggggcaccgagaccgtcagcaccaccctgcgctatggcttcttgctgctcatgaagcacccagaggtggagggtaaggctggagggggacggaagtggagggccccagaccctcaaaattccccttcgactggtgcaatgtccccacctgtcccagatcccgggaccctgagacgtgacttgctgtccagagacagggcaacattcagctggtaggcatcagctgagtctcattagatattaaaatattgaaaatgtctgcactgattggtcagtcacttctgtcccaagcccactgagtgcccactgcccgttccaccgggtcatcccctaagttcctccctgtgcctcccctgtgattctggcacaacctggttaacaggatcctactccaacaatgcgaatggctgatgtctgttctgttatgaatgctctacttccgtctcataggcggagccatatcatccaccccattttgcctattcggactatcatttcctgctctgagacccctagatacctaaacacattccccctcctcccccagccaaggtccatgaggagattgacagagtgatcggcaagaaccggcagcccaagtttgaggaccgggccaagatgccctacatggaggcagtgatccacgagatccaaagatttggagacgtgatccccatgagtttggcccgcagagtcaaaaaggacaccaagtttcgggatttcttcctccctaaggtgctatccgcccccaccccccagactacggggacttccagcccctctctgtgtccccagcatcccacccacattagaagctttctagaccctgtcccactccctcaatcagtcaaaaaagacttccccaaccaccacatccgttccacctttccacttagacactcctgagtcctgcatctctccagactctttgtgtcaggagaatcaaacacatgttcccaaacttcctatcttaagaaacagaagccccctttccattcggccttttgtcatagggacagaaatctcaggtcccccaaactcctgcctagaaggacatggaccccatgtctcccaaacttcctgtttcagagatgtgaaccttctatcccccaaagctcctccatcagaggaccccaactcctccatgcctgccacttccccttacctggggcacactagttccccctccagcccctgtgtactttcaccaatcccccgaccctcctcatcacacacaacttcctcctccctaccagggcaccgaagtgttccctatgctgggctctgtgctgagagaccccagtttcttctccaacccccaggacttcaatccccagcacttcctgaatgagaaggggcagtttaagaagagtgatgcttttgtgcccttttccatcggtaagagaccactgtttgctgccaggccacggctcacaccagcaggggcctccctcaccctcctcccctctctgcggtgtagcctggtatttctccagcttggaagttcctgttagaatctacccttgagccagcagctgatacttccttaactaccaagcacccagtacctgcgcccaggtaaaatgaaaggaaacatctttccccgtagatgtatttctctagggtcacacagcagattcctcagatccctaaaaaggagatgacggcacagcagtcatatttgcaagtgtacctggcaggaaaggacatctaaacctcccattgctacacctggcatggatcaccccatctatgatggatgtgtgacattatgcctttttcaaaacccatagaactgtataacacagagtaaaccctaatgtaaactatggactttgttagtaataatatatcaatattggttcaccattgttacatctcttatagaaagaaattgaggctcagggaggatcagagcctcctctgaaactctctcaggccataatattccacccttcctccctgggagagccgcagctggaggtcggtactggggcgaggctgcactgagagtgggcttcacctccacccctcccgcctctcctcctcaggaaagcggaactgtttcggagaaggcctggccagaatggagctctttctcttcttcaccaccgtcatgcagaacttccgcctcaagtcctcccagtcacctaaggacattgacgtgtcccccaaacacgtgggctttgccacgatcccacgaaactacaccatgagcttcctgccccgctgagcgagggctgtgccggtgcaggtctggtgggcggggccagggaaagggcagggccaagaccgggcttgggagaggggcgcagctaagactgggggcaggatggcggaaaggaaggggcgtggtggctagagggaagagaagaaacagaagcggctcagttcaccttgataaggtgcttccgagctgggatgagaggaaggaaacccttacattatgctatgaagagtagtaataatagcagctcttatttcctgagcacgtacccccgtgtcacctttgttcaaaaaccattgcacgctcacctaattgccacaaacctctgcgaagggcgttcatgcccattttacacgtgacaaaactgaggcttagaaagttgtctctgatgtctcacaaaacataagtgcccagaaaatctttgaacacagatctgtgcccatagccttctagacagattcttaaaaagcacctattcctcacgtaaaacagcttagtatagcatcacatggcctgaacatccctgtcctggggagttttccagagacctggcgggtggctgtcctgccttcactgcacacatgcccacactctcacctactcaacatgctttgaatacccgggtgtaatctgtgcttgctaccagataaggccactgtagcccattcagagtcagtccagggacacaacgagacatgaatggacatacagagtcagtccattaacaattttttgcagagcagaagtttttaattttaatgacattctgtcaatatatcccttaatgaagaagttgaaggaaagaatcacttacagagaatgagagaactctggatcaggacatagccatattcatgtgtgtgatcatgttcagacatgcatgtgagcatatgactgcatattctcctccgtgtatgcatgaaacatattccactatgggttctgaaggctgggctctcctgatggatctggggtctgttgtaagacccagctgagagtttcacaatcactgcccactctcaaagaagttagtgtgtgtcccccttagaagctcccttacggctttcacgaaaattaactccaagtttattgtagacctaaatgaatagtgagtacaaaacttctagaagataatgtaggagaaaatctagatgaccttaagtttggtgatggcttattagatacaacaccaaaagcacaatccttgaaagaagcaattgataagttctgtgtcattaaaattagaagcttctgctctgcaaaagaattgtcaagagaatgacaatacaagctacagactaggagaacatatttgcaggggacatacctgataagggacttacattcaaaatacacaaagaactgttaaaactcaatagaaaacaacccaattaaagaatgggcaaacgatctgagcagacacctcaccaaagaaaatatacacatagcaaataagcataaggaaagatgctccatatcatatgtcagtagccatttcaaattgaaacaataatgatgtaccactactcacctattagaatggctaaaatccaaagctgacaacatcaaatgctgacaaggaggtggctcaacaggaactgtcattcattgatggtgggaatgcaaaatgacacagctactttgaaagacaatttggcggtttcttgcaaagctaaacatattcttaccatgtgctacagcaatcatgttcttcggtattaacccaaatgagttgaaaacatatagcaacacaaaaacctctgcacagatgtttatagcagctttattcataattgccaaatcttgtaagcaaccaagatgttcttcaacaagtgaatggataaatgaactgtaatatgttcatacaatgaaatacggcagggcttgatggctcatactgttaattatagcactatgggaggccgaggtgggcagatcacttgaggtcaggaattcgagaccagcctggccaacatggtgaaaccccatctctactaaaaatacaaaaattagccgggcacggtggcatgcgcctgcagttgcagccacttgggaggctgaggtgggagaattgcctgaacccataaggcagaggttgcagcgagctgagatggagccactgcactccagcctgggcgacacagtgagattagaagaagcaatattgttaaaatgttcatactactcaaagcaatctacagattcaatgcaattcctatcaaaataccaatgacattcttcacagaaacagaaaaaa

(19) United States
(12) Patent Application Publication
(10) Pub. No.: US 2009/0305249 A1
(43) Pub. Date: Dec. 10, 2009

(54) METHOD OF DETECTING CYP2A6 GENE VARIANTS

(75) Inventors: Ben P. Anderson, Aliso Viejo, CA (US); Feras M. Hantash, Dana Point, CA (US); Weimin Sun, Irvine, CA (US)

Correspondence Address: FOLEY & LARDNER LLP, P.O. BOX 80278, SAN DIEGO, CA, 92138-0278, US

(73) Assignee: Quest Diagnostics Investments Incorporated

(21) Appl. No.: 12/116,879

(22) Filed: May 7, 2008

(51) Int. Cl. C12Q 1/68

(52) U.S. Cl. 435/6

(57) ABSTRACT

The present invention relates to methods for amplifying various regions of the CYP2A6 gene. Methods are provided for amplifying one or more fragments of the CYP2A6 gene in a single tube. The methods can identify mutations, deletion, duplication, and/or rearrangement in a sample containing the CYP2A6 gene.

### FIELD OF THE INVENTION

The invention relates generally to the field of diagnostic assays, in particular, assays for identifying mutations, deletion, duplication, and/or rearrangement in the CYP2A6 gene.

### BACKGROUND OF THE INVENTION

The following description is provided to assist the understanding of the reader. None of the information provided or references cited is admitted to describe or constitute prior art to the present invention.

The cytochrome P450 enzymes comprise a superfamily of monooxygenases that are involved with the metabolism of many endogenous and exogenous substances. Polymorphisms in these metabolizing enzymes are often responsible for the inter-individual differences that are seen in the responses to drugs and chemicals. Cytochrome P450 2A6 (CYP2A6) is expressed mainly in the liver and represents about 1-10% of the total liver P450 protein. CYP2A6 plays a major role in the oxidation of nicotine and coumarin. Polymorphisms in the CYP2A6 gene can contribute to nicotine dependence and cancer susceptibility. CYP2A6 is also responsible for metabolizing several classes of neuroactive drugs including medications for Alzheimer's disease and Attention Deficit Disorder.

Nucleic acid sequencing refers to biochemical methods of determining the order of the nucleotide bases, adenine, guanine, cytosine, and thymine, in an polynucleotide. Determining the nucleic acid sequence is useful in basic research studying fundamental biological processes, as well as in applied fields such as diagnostic or forensic research. The advent of DNA sequencing has significantly accelerated biological research and discovery. The rapid speed of sequencing attainable with modern DNA sequencing technology has been instrumental in the large-scale sequencing of the human genome and DNA sequencing has become increasingly important in molecular diagnostics.

### SUMMARY OF THE INVENTION

Provided are methods for detecting variants (e.g., mutations, deletion, duplication, and/or rearrangement) in the CYP2A6 gene.

In one aspect, methods for determining the presence or absence of one or more mutations in the CYP2A6 gene from nucleic acids in a sample include using at least one oligonucleotide capable of hybridizing to one or more of the sequences of SEQ ID NOs: 1-10 or complements thereof to amplify at least one fragment of the CYP2A6 gene, containing the site of the mutation, and detecting the presence or absence of one or more mutations in the CYP2A6 gene in which at least one oligonucleotide suitable for hybridizing and amplifying is capable of specifically amplifying the CYP2A6 gene but not the CYP2A7 gene or pseudogenes of CYP2A6 and CYP2A7. In certain preferred embodiments, at least one oligonucleotide suitable for hybridizing and amplifying comprises at least one of the sequences of SEQ ID NOs: 1′-20 or complements thereof, with a proviso that the 5′ end of the sequences of SEQ ID NOs: 11-20 and complements thereof comprises 1 to 6 optional nucleotides as shown in Table 2. For example, any of the sequences of SEQ ID NOs: 1-20 may comprise the full-length sequence as shown in Table 2 or may comprise a sequence with 1, 2, 3, 4 or 5 fewer nucleotides on the 5′ end of the sequences as long as the sequences specifically amplify the CYP2A6 gene but not the CYP2A7 gene or pseudogenes of CYP2A6 and CYP2A7. The optional nucleotides are shown in bold and underlined in Table 2. In certain preferred embodiments, at least one oligonucleotide suitable for hybridizing and amplifying comprises a sequence selected from the sequences of SEQ ID NOs: 21-30 and complements thereof. In a preferred embodiment, amplifying is accomplished with polymerase chain reaction (PCR). In a preferred embodiment, the method further includes performing single nucleotide primer extension to detect the identity of the nucleotide added to the extension primer, in which the identity of the nucleotide indicates the presence or absence of the mutation in the CYP2A6 gene. In a preferred embodiment, the method further includes detecting the presence of one or more mutations by separating the reaction product(s) of single nucleotide primer extension by size and by detectable moiety. In a related preferred embodiment, the detectable moiety is fluorescently labeled. In certain preferred embodiments, the single nucleotide extension primer includes a labeled ddNTP. In related preferred embodiments, the labeled ddNTP is fluorescently labeled. In preferred embodiments, two or more fragments are amplified, preferably in the same vessel. In certain preferred embodiments, amplifying is accomplished with a multiplex polymerase chain reaction (PCR). In some preferred embodiments, the single nucleotide primer extension includes extension primers selected from the sequences of SEQ ID NOs: 41-59.

In a second aspect, methods for detecting gene deletion, duplication, and/or rearrangement in the CYP2A6 gene from nucleic acids in a sample include using at least one oligonucleotide capable of hybridizing to one or more of the sequences of SEQ ID NOs: 1-4, 7-10 or complements thereof to amplify at least one fragment of the CYP2A6 gene, containing the suspected gene deletion, duplication, and/or rearrangement, and detecting the deletion, duplication, and/or rearrangement in the CYP2A6 gene using dosage analysis, in which a substantial decrease or increase in the amount of detectable fragment observed indicates a deletion, duplication, and/or rearrangement of the CYP2A6 gene, in which at least one oligonucleotide suitable for hybridizing and amplifying is capable of specifically amplifying the CYP2A6 gene but not the CYP2A7 gene or pseudogenes of CYP2A6 and CYP2A7. In a preferred embodiment, at least one oligonucleotide suitable for amplifying the fragment includes a sequence selected from the sequences of: SEQ ID NOs: 22, 24, 28, 29, 37, 38, 39, 40 and complements thereof. In a preferred embodiment, one or more oligonucleotides is detectably labeled, more preferably fluorescently labeled. In certain preferred embodiments, FAM fluorescent dyes are present on different oligonucleotides and associated with the resulting amplicons. In preferred embodiments, two or more fragments are amplified, preferably in the same vessel. In certain preferred embodiments, amplifying is accomplished with a multiplex polymerase chain reaction (PCR).

In a preferred embodiment, amplification also includes at least one or more oligonucleotides suitable for amplifying at least one internal control fragment that does not correspond to the CYP2A6 gene. In a preferred embodiment, the oligonucleotide(s) is selected from the sequences of SEQ ID NOs: 31-36 and complements thereof. In a preferred embodiment, at least three internal control fragments are amplified. In a preferred embodiment, the internal controls can be segments of various genes. Such segments can include an exon from the CYP2A7 gene, an exon from the CFTR gene, and/or an exon from the CYP1B1 gene. Other internal controls can be used.

In accordance with particular embodiments of the above aspects, amplification is performed without the aid of complex PCR methods such as nested PCR, touchdown PCR, allelic-specific PCR, gap PCR, or RFLP pattern differentiation.

In various embodiments of the methods, one or more steps are performed in an on-line automated fashion. One or more of the steps of the assays described herein are preferably performed in an automated fashion, typically using robotics, in order to provide for the processing of a large number of samples in a single batch run. Preferred forms of automation will provide for the preparation and separation of a plurality of labeled nucleic acids, preferably in small volumes.

In preferred embodiments of the above aspects, there are provided primers capable of hybridizing and specifically amplifying portions of the CYP2A6 gene but not the CYP2A7 gene or pseudogenes of CYP2A6 and CYP2A7. Preferably primers are 12-55 nucleotides in length; preferably 14-40 nucleotides; preferably 15-35 nucleotides; or more preferably 16-30 nucleotides. Preferred primers include sequences from SEQ ID NOs: 21-30.

In still another embodiment there are provided extension primers that are useful for detecting the CYP2A6 mutations. Accordingly, provided are substantially purified nucleic acids comprising 15-30 nucleotides complementary to a segment of the CYP2A6 gene that is upstream of a mutation site and terminating one nucleotide 5′ of that mutation site. Suitable extension primers are described herein, and can be one of the sequences set forth in SEQ ID NOs: 41-59.

The present CYP2A6 assay as described in the above aspects can detect one or more of the exons of the CYP2A6 gene. In some embodiments, the method can be used to detect at least 3, at least 4, or at least 5 fragments in a single multiplex PCR, the fragments representing at least 2, at least 3, at least 4, at least 5, at least 6, or at least 7 different exons of the CYP2A6 gene.

Kits comprising oligonucleotides for performing amplifications as described herein also are provided.

As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

As used herein, the term “sample” or “test sample” includes clinical samples, isolated nucleic acids, or isolated microorganisms. In preferred embodiments, a sample is obtained from a biological source (i.e., a “biological sample”), such as tissue, bodily fluid, or microorganisms collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). The term “patient sample” as used herein refers to a sample obtained from a human seeking diagnosis and/or treatment of a disease.

As used herein, the term “cell sample” includes any source of cells containing nucleic acids that are to be used as a template in a nucleic acid amplification reaction. Cells can be prokaryotic or eukaryotic. Eukaryotic cell samples can be animal or plant cells. Preferred eukaryotic cell samples are mammalian cells, preferably human. In some embodiments, a cell sample can be cells in culture or a tissue sample from an animal, most preferably, a human. Tissue samples include, but are not limited to, blood, bone marrow, cell-containing body fluids such as cerebrospinal fluid, or tissue (e.g., biopsy material). Preferred samples include whole blood. Cell samples can be packed cells or cells suspended in liquid.

The terms “body fluid” or “bodily fluid” are used interchangeably herein and refer to a fluid sample from a human or other animal. Body fluids include, but are not limited to amniotic fluid, cerebrospinal fluid, peritoneal fluid, pleural fluid, synovial fluid, pericardial fluid, intraocular fluid, bronchial alveolar lavage (BAL), bronchial wash (BW), blood, plasma, serum, saliva, mucus, semen, sputum, tears, and urine. Body fluids can be cell-containing or acellular.

As used herein “acellular body fluid” refers to a body fluid lacking cells. Such acellular body fluids are generally produced by processing a cell-containing body fluid by, for example, centrifugation or filtration, to remove the cells. Acellular body fluid, however, can contain cell fragments or cellular debris. Preferred acellular body fluids are plasma and serum.

As used herein the term “extracted” used in reference to nucleic acids in a cell sample means that the nucleic acids have been physically separated from cells containing the nucleic acid by addition of a sufficient volume of organic solvent to lyse the cells and separate the protein from the nucleic acids, wherein the nucleic acid in the aqueous phase is separated from the protein. The nucleic acids in the aqueous phase can be concentrated by addition of a sufficient volume of ethanol to precipitate the nucleic acids. Other methods of extracting nucleic acids include the capture of nucleic acids on solid phase using, for example, an oligonucleotide-coupled bead (e.g., oligo-dT).

The term “small volumes” as used herein refers to volumes of liquids less than 5 mL, e.g., any volume from about 0.001 μL, to any volume about 5 mL, 3 mL, 2 mL, 500 μL, 200 μL, 100 μL, 10 μL, 1 μL, 0.1 μL, 0.01 μL, or 0.001 μL.

As used herein, “nucleic acid” refers broadly to genomic DNA, segments of a chromosome, segments or portions of DNA, cDNA, and/or RNA. Nucleic acid can be derived or obtained from an originally isolated nucleic acid sample from any source (e.g., isolated from, purified from, amplified from, cloned from, reverse transcribed from sample DNA or RNA). As used herein, “genomic nucleic acid” or “genomic DNA” refers to some or all of the DNA from the nucleus of a cell. Genomic DNA can be intact or fragmented (e.g., digested with restriction endonucleases by methods known in the art). In some embodiments, genomic DNA includes sequence from all or a portion of a single gene or from multiple genes, sequence from one or more chromosomes, or sequence from all chromosomes of a cell. In contrast, the term “total genomic nucleic acid” is used herein to refer to the full complement of DNA contained in the genome of a cell. As is well known, genomic nucleic acid includes gene coding regions, introns, 5′ and 3′ untranslated regions, 5′ and 3′ flanking DNA and structural segments such as telomeric and centromeric DNA, replication origins, and intergenic DNA. Genomic nucleic acid can be obtained from the nucleus of a cell, or recombinantly produced. Genomic DNA can also be transcribed from DNA or RNA isolated directly from a cell nucleus. PCR amplification can also be used. Methods of purifying DNA and/or RNA from a variety of samples are well-known in the art.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose. RNA can be used in the methods described herein and/or can be converted to cDNA by reverse-transcription for use in the methods described herein. Methods for reverse transcription are well known in the art. See, e.g., See Maniatis et al., Molecular Cloning, A Laboratory Manual, 2d, Cold Spring Harbor Laboratory Press, page 16-54 (1989).

As used herein, the term “oligonucleotide” refers to a short polymer composed of deoxyribonucleotides, ribonucleotides or any combination thereof. Oligonucleotides are generally between about 10, 11, 12, 13, 14 or 15 to about 150 nucleotides in length, more preferably about 10, 11, 12, 13, 14, or 15 to about 70 nucleotides, and most preferably between about 12 to about 60 nucleotides in length. The single letter code for nucleotides is as described in the U.S. Patent Office Manual of Patent Examining Procedure, section 2422, table 1. In this regard, the nucleotide designation “R” means purine such as guanine or adenine, “Y” means pyrimidine such as cytosine or thymidine (uracil if RNA); and “M” means adenine or cytosine. An oligonucleotide can be used as a primer or as a probe.

The term “sense strand” as used herein means the strand of double-stranded DNA (dsDNA) that includes at least a portion of a coding sequence of a functional protein. As used herein “anti-sense strand” means the strand of dsDNA that is the reverse complement of the sense strand.

The term “coding sequence” as used herein means a sequence of a nucleic acid or its complement, or a part thereof, that can be transcribed and/or translated to produce the mRNA for and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the encoding sequence can be deduced therefrom.

The term “non-coding sequence” as used herein means a sequence of a nucleic acid or its complement, or a part thereof, that is not transcribed into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, etc.

As used herein the term “pseudogene” means a nonfunctional gene sequence that is related to a known gene but cannot be transcribed or translated, due to mutations. The term “missense mutations” or “nonsynonymous mutations” as used herein describes types of point mutations where a single nucleotide is changed to cause substitution of a different amino acid. Not all missense mutations lead to appreciable protein changes. An amino acid can be replaced by an amino acid of very similar chemical properties, in which case, the protein will still function normally; this is termed a neutral or “quiet” mutation. When an amino acid is encoded by more than one codon (so-called “degenerate coding”), such a mutation in a codon will not produce any change in translation; this is a synonymous mutation and not a missense mutation.

As used herein, a “mutation” is at least a single nucleotide variation (i.e., a “point mutation”) in a nucleic acid sequence relative to the normal sequence or wild-type sequence. A mutation can include a substitution, a deletion, an inversion or an insertion. With respect to an encoded polypeptide, a mutation can be “silent” and result in no change in the encoded polypeptide sequence or a mutation can result in a change in the encoded polypeptide sequence. For example, a mutation can result in a substitution in the encoded polypeptide sequence. A mutation can result in a frameshift with respect to the encoded polypeptide sequence. A “mutant” can include a nucleic acid having at least one mutation. The “mutation site” refers to the location in the nucleic acid where the mutation can be found, when present.

In a normal diploid eukaryote, each gene has 2 loci, i.e., 1 gene copy at the same locus (position) on each chromosome of a chromosome pair. Different versions of a gene can occur at any locus, and these versions are called alleles. Each allele can be the wild-type (normal) allele or an allelic variant. Thus, two different versions of a CYP2A6 gene can be present in any particular subject. The term “allelic variant” as used herein refers to a mutation or variation in a nucleotide sequence, such as a single nucleotide polymorphism (SNP) or any other variant nucleic acid sequence or structure (e.g., duplications, deletions, inversions, insertions, translocations, etc.) in a gene encoding a gene that alters the activity and/or expression of the gene. Allelic variants can over- or under-express the polypeptide encoded by the gene, and/or can express proteins altered activities by virtue of having amino acid sequences that vary from wildtype sequence.

A “single nucleotide polymorphism”, or “SNP” as used herein, is a DNA sequence variation occurring when a single nucleotide—A, T, C, or G—in the genome (or other shared sequence) differs between members of a species (or between paired chromosomes in an individual). For example, two sequenced DNA fragments from different individuals, AAGCCTA to AAGCTTA, contain a difference in a single nucleotide. Almost all common SNPs have only two alleles. Within a population, SNPs can be assigned a minor allele frequency—the ratio of chromosomes in the population carrying the less common variant to those with the more common variant. A SNP allele that is common in one geographical or ethnic group can be much rarer in another.

Single nucleotide polymorphisms can fall within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed synonymous (sometimes called a silent mutation)—if a different polypeptide sequence is produced they are non-synonymous. SNPs that are not in protein-coding regions can still have consequences for gene splicing, transcription factor binding, or the sequence of non-coding RNA.

As used herein, “dideoxynucleotides”, or ddNTPs, are nucleotides lacking a 3′-hydroxyl (—OH) group on their deoxyribose sugar. Since deoxyribose already lacks a 2′-OH, dideoxyribose lacks hydroxyl groups at both its 2′ and 3′ carbons. The lack of this hydroxyl group means that, after being added by a DNA polymerase to a growing nucleotide chain, no further nucleotides can be added as no phosphodiester bond can be created based on the fact that deoxyribonucleoside triphosphates allow DNA chain synthesis to occur through a condensation reaction between the 5′ phosphate (following the cleavage of pyrophospate) of the current nucleotide with the 3′ hydroxyl group of the previous nucleotide. The dideoxyribonucleotides do not possess a 3′ hydroxyl group, hence no further chain elongation can occur once this dideoxynucleotide is on the chain. This can lead to the determination of the DNA sequence. Thus, these molecules form the basis of the dideoxy chain-termination method of DNA sequencing, which was developed by Frederick Sanger in 1977.

In certain embodiments of the above aspects of the invention, more than one labeled ddNTP is used. In this case, the ddNTPs can be distinguishably labeled. “Distinguishably labeled,” means that each type of member of a set is labeled with a label that can be distinguished from the label(s) used for other members of the set. In some aspects of the invention, the distinguishable label is a fluorescent label. For example, in a set of distinguishably labeled nucleotides (e.g., dideoxy NTPs, or ddNTPs), each type of nucleotide is labeled with a label that can be distinguished from the labels of the other types of nucleotides. Thus, for example, if four labels designated *1, *2, *3 and *4 are used to label the four types of ddNTPs, each ddATP molecule can carry label *1 each ddTTP molecule can carry label *2, each ddCTP molecule can carry label *3, and each ddGTP molecule can carry label *4. If, for example, the mutant nucleotide in a particular sequence is an adenine, when the mutation is present, only dd(T*2)TP can be added to the 3′ end of the extension primer for the amplified region containing the mutation, because thymine (T) is the only base that pairs with adenine (A). The addition of the dd(T*2)TP to the 3′ of the primer prevents any further primer extension because it is a dideoxynucleotide, also known as a chain-terminating nucleotide. Thus, the only primer that is 3′ extended on the mutant amplified region is labeled with label *2. Detection of the signal from label *2 indicates that the mutation (i.e., adenine) is present in the sample. If the tested DNA is from an individual that is heterozygous at that position, then the signal from a second label (i.e., *1, *3 or *4) will be detected as well.

As used herein, a “primer” for amplification is an oligonucleotide that is complementary to a target nucleotide sequence and leads to addition of nucleotides to the 3′ end of the primer in the presence of a DNA or RNA polymerase. The 3′ nucleotide of the primer should generally be identical to the target sequence at a corresponding nucleotide position for optimal expression and/or amplification. The term “primer” as used herein includes all forms of primers that can be synthesized including peptide nucleic acid primers, locked nucleic acid primers, phosphorothioate modified primers, labeled primers, and the like.

As used herein, a “forward primer” is a primer that anneals to the anti-sense strand of dsDNA. A “reverse primer” anneals to the sense-strand of dsDNA. A “primer pair” refers to the combination of a forward primer and a reverse primer, each specific for the same target nucleic acid or fragment.

The term “target nucleic acid” or “target sequence” as used herein refers to a sequence which includes a segment of nucleotides of interest to be amplified and detected. Copies of the target sequence which are generated during the amplification reaction are referred to as amplification products, amplimers, or amplicons. Target nucleic acid can be composed of segments of a chromosome, a complete gene with or without intergenic sequence, segments or portions of a gene with or without intergenic sequence, or sequences of nucleic acids for which probes or primers are designed. Target nucleic acids can include a wild-type sequence(s), a mutation, a deletion, a rearrangement, a duplication, tandem repeat regions, a gene of interest, a region of a gene of interest, or any upstream or downstream region thereof. Target nucleic acids can represent alternative sequences or alleles of a particular gene. Target nucleic acids can be derived from genomic DNA, cDNA, or RNA. As used herein target nucleic acid can be DNA or RNA extracted from a cell or a nucleic acid copied or amplified therefrom, or can include extracted nucleic acids further converted using a bisulfite reaction.

The term “region” or “fragment” as used herein in reference to a gene, refers to a piece of contiguous nucleic acid. In certain embodiments, a fragment includes one or more mutation sites, preferably one or more mutation sites within the CYP2A6 gene. In preferred embodiments a fragment containing one or more mutation sites is amplified. In such embodiments the fragment contains at least 40 nucleotides, preferably at least 50 nucleotides, preferably at least 75 nucleotides, preferably at least 100 nucleotides, preferably at least 200 nucleotides, preferably at least 500 nucleotides, or more preferably at least 700 nucleotides. In certain embodiments, the fragment can be 1000, 2000, 3000, or even 5000 nucleotides, provided that the DNA strands can be separated and that the extension primer and polymerase are able to hybridize and extend. In particular examples, amplification can include amplifying portions of nucleic acids between about 30 and 50, between about 50 and 100, between about 100 and 500, between about 500 to 700, or between about 700 and 1300 nucleotides in length; for example, in some preferred embodiments, amplification products may be between about 740 to about 1260 nucleotides in length. The length of the amplicon can be pre-determined by selecting the proper primer sequences. A fragment can include one or more exons of the CYP2A6 gene.

In certain embodiments, the fragments can be used in polymerase chain reaction (PCR), various hybridization procedures, or microarray procedures to identify or amplify identical or related parts of mRNA or DNA molecules. A fragment or segment can uniquely identify each polynucleotide sequence of the methods described herein.

Variations in the DNA sequences of humans can affect how humans develop diseases and respond to pathogens, chemicals, drugs, vaccines, and other agents. However, their greatest importance in biomedical research is for comparing regions of the genome between cohorts in clinical trials (such as with matched cohorts with and without a disease). Assays detecting gene variants can be used to predict toxicity, adjust dosing, stratify patients into dosing levels, and exclude poor metabolizers.

The phrase “comprise sequence from all or a portion of” as used herein in reference to an exon means that the sequence represents all of the exon or at least 10 bases of the exon. In other embodiments, most of the exon is amplified, generally greater than 50%, greater than 60%, greater than 70%, greater than 80%, greater than 90% and greater than 95%.

As used herein, the term “substantially identical”, when referring to a nucleic acid, is one that has at least 80%, at least 85%, at least 90%, at least 95%, or at least 99% sequence identify to a reference nucleic acid sequence. The length of comparison is preferably the full length of the nucleic acid, but is generally at least 12 nucleotides, at least 15 nucleotides, 20 nucleotides, 30 nucleotides, 40 nucleotides, 50 nucleotides, 75 nucleotides, 100 nucleotides, 125 nucleotides, or more.

As used herein, the term “purified” in reference to oligonucleotides does not require absolute purity. Instead, it represents an indication that the sequence is relatively more pure than in the natural environment. Such oligonucleotides can be obtained by a number of methods including, for example, laboratory synthesis, restriction enzyme digestion or PCR. A “purified” oligonucleotide is preferably at least 10% pure. A “substantially purified” oligonucleotide is preferably at least 50% pure, more preferably at least 75% pure, and most preferably at least 95% pure. The nucleic acid sample can exist in solution or as a dry preparation.

By “isolated”, when referring to a nucleic acid (e.g. an oligonucleotide) as used herein is meant a nucleic acid that is apart from a substantial portion of the genome in which it naturally occurs. For example, any nucleic acid that has been produced synthetically (e.g., by serial base condensation) is considered to be isolated. Likewise, nucleic acids that are recombinantly expressed, produced by a primer extension reaction (e.g., PCR), or otherwise excised from a genome are also considered to be isolated.

The term “complement” “complementary” or “complementarity” as used herein with reference to polynucleotides (i.e., a sequence of nucleotides such as an oligonucleotide or a target nucleic acid) refers to standard Watson/Crick pairing rules.

The “complement” of a nucleic acid sequence as used herein refers to an oligonucleotide which, when aligned with nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in natural nucleic acids can be included in the nucleic acids described herein; these include, for example, inosine, 7-deazaguanine, locked nucleic acids (LNA), and peptide nucleic acids (PNA). Complementarity need not be perfect; stable duplexes can contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be a sequence of RNA complementary to the DNA sequence or its complement sequence, and can also be a cDNA.

The term “substantially complementary” as used herein means that two sequences specifically hybridize (defined below). The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

As used herein, an oligonucleotide is “specific” for a nucleic acid if the oligonucleotide has at least 50% sequence identity with a portion of the nucleic acid when the oligonucleotide and the nucleic acid are aligned. An oligonucleotide that is specific for a nucleic acid also is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity. Sequence identity can be determined using a commercially available computer program with a default setting that employs algorithms well known in the art (e.g., BLAST). As used herein, sequences that have “high sequence identity” have identical nucleotides at least at about 50% of aligned nucleotide positions, preferably at least at about 60% of aligned nucleotide positions, and more preferably at least at about 75% of aligned nucleotide positions.

As used herein, an oligonucleotide (e.g., a primer) that is specific for a target nucleic acid or fragment will “hybridize” to the target nucleic acid or fragment under suitable conditions. As used herein, “hybridization” or “hybridizing” refers to the process by which an oligonucleotide single strand anneals with a complementary strand through base pairing under defined hybridization conditions. Hybridizations are typically and preferably conducted with nucleic acid molecules, preferably 20-100 nucleotides in length, more preferably 15-60 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

“Specific hybridization” as used herein is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art. Stringency of hybridization can be expressed, in part, with reference to the temperature under which the wash steps are carried out. Such temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Equations for calculating Tₘ and conditions for nucleic acid hybridization are known in the art.

Oligonucleotides used as primers or probes for specifically amplifying (i.e., amplifying a particular target nucleic acid or fragment sequence) or specifically detecting (i.e., detecting a particular target nucleic acid or fragment sequence) a target nucleic acid or fragment generally are capable of specifically hybridizing to the target nucleic acid or fragment.

The term “flanking” as used herein means that an oligonucleotide hybridizes to a target nucleic acid or fragment adjoining a region of interest sought to be amplified on the target. The skilled artisan will understand that preferred oligonucleotides hybridize upstream from a region of interest, on a strand of a target double-stranded DNA molecule, such that nucleotides can be added to the 3′ end of the primer by a suitable DNA polymerase. Primers that flank a CYP2A6 exon are generally designed not to anneal to the exon sequence but rather to anneal to a sequence that adjoins the exon (e.g., intron sequence). However, in some cases, amplification oligonucleotides can be designed to anneal to the exon sequence. The location of oligonucleotide annealing for the oligonucleotides that can be used with the methods is shown in Table 1.

The term “suitable for amplifying,” when referring to a primer, as used herein describes oligonucleotides that specifically hybridize to a target nucleic acid or fragment and are capable of providing an initiation site for a primer extension reaction in which a complementary copy of the target nucleic acid or fragment is synthesized.

The term “amplification” or “amplify” as used herein with respect to nucleic acid sequences includes methods for copying a target nucleic acid or fragment, thereby increasing the number of copies of a selected nucleic acid sequence. Amplification can be exponential or linear. A target nucleic acid or fragment can be either DNA or RNA. The sequences amplified in this manner form an “amplicon” or “amplification product”. While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods can be used either in place of, or together with, PCR methods. See, e.g., Saiki, “Amplification of Genomic DNA” in PCR Protocols, Innis et al., Eds., Academic Press, San Diego, Calif. 1990, pp 13-20; Wharam, et al., Nucleic Acids Res. 2001 Jun. 1; 29(11):E54-E54; Hafner, et al., Biotechniques 2001 April; 30(4):852-6, 858, 860; Zhong, et al., Biotechniques 2001 April; 30(4):852-6, 858, 860.

“Multiplex format” refers generally to the case in which two or more independent reactions occur simultaneously in a single reaction vessel. As used herein, certain steps in the methods described herein can be performed in multiplex format, including, for example, amplification or single nucleotide primer extension. Thus, as used herein a “multiplex amplification reaction” (e.g., a multiplex PCR reaction) refers to a PCR reaction where more than one primer set is included in the reaction mixture, allowing two or more different regions to be amplified by the PCR in a single reaction vessel (e.g., in a tube or in a well of a microtiter plate).

By “primer extension reaction” as used herein is meant a synthetic reaction in which a primer hybridizes to a target nucleic acid or fragment and a complementary copy of the target nucleic acid or fragment is produced by the polymerase-dependent 3′-addition of individual complementary nucleotides. In preferred embodiments, the primer extension reaction is PCR. Primer extension refers to the enzymatic addition of at least one nucleotide to the three-prime (3′) hydroxy group of an extension primer, which is an oligonucleotide that is paired to a template nucleic acid (for an example of primer extension as applied to the detection of polymorphisms, see Fahy et al., Multiplex fluorescence-based primer extension method for quantitative mutation analysis of mitochondrial DNA and its diagnostic application for Alzheimer's disease, Nucleic Acid Research 25:3102-3109, 1997). The extension reaction is catalyzed by a DNA polymerase. Primer extension reactions can be “single base extensions” where the primer is extended by a single base.

Single base extension is a method for determining the identity of a nucleotide base at a specific position along a nucleic acid. The method is used to identify a single nucleotide polymorphism (SNP). In the method, a primer hybridizes to a complementary region along the nucleic acid, to form a duplex, with the primer's terminal 3′ end directly adjacent to the nucleotide base to be identified. The primer is enzymatically extended a single base by a nucleotide terminator complementary to the nucleotide being identified. The terminator prevents additional nucleotides from being incorporated. Many different approaches can be taken for determining the identity of a terminator, including but not limited to, fluorescence labeling, mass labeling for mass spectrometry, measuring enzyme activity using a protein moiety, and isotope labeling.

By “DNA polymerase” is meant a DNA polymerase, or a fragment thereof, that is capable of carrying out primer extension. Thus, a DNA polymerase can be an intact DNA polymerase, a mutant DNA polymerase, an active fragment from a DNA polymerase, such as the Klenow fragment of E. coli DNA polymerase, and a DNA polymerase from any species including, but not limited to, thermophiles.

Addition of one or more nucleotides to the 3′ end of the extension primer generates an oligonucleotide having a length greater than the extension primer. The extended oligonucleotide, therefore, has a length of at least (X+Y) nucleotides, where X is the length of the extension primer and Y is the number of bases added to the extension primer by the polymerase. If one of the nucleotides in the added sequence Y is labeled, then the extended (X+Y) oligonucleotide is labeled. In preferred embodiments, the nucleotide added is in the form of a dideoxynucleotide. Thus, extension of the primer is terminated with the addition of a single nucleotide.

As used herein, the term “terminator” or “chain terminating nucleotide” refers to a nucleotide, nucleotide-based nucleotide analog, or acyclo-based analog capable of being added to the terminus of a nucleic acid primer and further capable of specific base-pairing with a nucleotide present in a complementary nucleic acid and which prevents further chain elongation after incorporation at the terminus of a nucleic acid chain. Exemplary terminators include 2′,3′-dideoxynucleotides such as ddATP, ddGTP, ddCTP and ddTTP. Analogs of 2′,3′-dideoxynucleotide terminators are also included, for example, 5-bromo-dideoxyuridine, 5-methyl-dideoxycytidine and dideoxyinosine are suitable analogs. Other 3′-deoxynucleoside analogs can also be used as terminator nucleotides.

The term “multiplex primer extension reaction” as used herein refers to a primer extension reaction that is capable of simultaneously producing complementary copies of two or more target nucleic acids or fragments within the same reaction vessel. Each reaction product is primed using distinct oligonucleotides. A multiplex reaction can further include specific probes for each product that are detectably labeled with different detectable moieties. In preferred embodiments, the multiplex primer extension reaction is a multiplex PCR in which two or more products within the same reaction vessel are amplified.

The term “single nucleotide primer extension reaction” or “SNaPshot” (Lindblad-Toh et al., Nature Genetics 24 (2000) 381-6) as used herein refers to a primer extension reaction in which a primer, designed to end one nucleotide short of a specific mutation site, hybridizes to the PCR amplicon in the presence of fluorescently labeled ddNTPs (without dNTPs) and a DNA polymerase. The polymerase extends the primer by one nucleotide, adding a single fluorescently labeled ddNTP to its 3′ end. Each di-deoxynucleotide is labeled with a different fluorescent colored dye. The SNaPshot primers can be designed with different lengths to allow detection of multiple mutations in a single SNaPshot reaction. Excess ddNTPs in the SNaPshot reaction mixture can be treated with Calf Intestinal Phosphatase (CIP) to prevent interference of the un-incorporated dNTPs in the detection step.

As used herein, the term “on-line” or “inline”, for example as used in “on-line automated fashion” or “on-line extraction” refers to a procedure performed without the need for operator intervention. In contrast, the term “off-line” as used herein refers to a procedure requiring manual intervention of an operator.

As used herein, the term “detecting” used in the context of detecting a signal from a detectable label to indicate the presence of a target nucleic acid or fragment in the sample does not require the method to provide 100% sensitivity and/or 100% specificity. As is well known, “sensitivity” is the probability that a test is positive, given that the subject has a target nucleic acid or fragment sequence, while “specificity” is the probability that a test is negative, given that the subject does not have the target nucleic acid or fragment sequence. A sensitivity of at least 50% is preferred, although sensitivities of at least 60%, at least 70%, at least 80%, at least 90% and at least 99% are clearly more preferred. A specificity of at least 50% is preferred, although sensitivities of at least 60%, at least 70%, at least 80%, at least 90% and at least 99% are clearly more preferred. Detecting also encompasses assays with false positives and false negatives. False negative rates can be 1%, 5%, 10%, 15%, 20% or even higher. False positive rates can be 1%, 5%, 10%, 15%, 20% or even higher.

The phrase “detectable moiety” as used herein denotes any molecule (or combinations of molecules) that can be attached or otherwise associated with a molecule so that the molecule can be detected indirectly by detecting the detectable moiety. A detectable moiety can be a radioisotope (e.g., iodine, indium, sulfur, hydrogen etc.) a dye or fluorophore (e.g., cyanine, fluorescein, rhodamine), protein (e.g. avidin, antibody), enzyme (peroxidase, phosphatase, etc.), or any other agent that can be detected directly or indirectly. An enzyme is an example of a detectable moiety detected by indirect means. In this case, the enzyme is attached to the target nucleic acid and the presence of the enzyme is detected by adding an appropriate substrate that when acted upon by the enzyme, causes the substrate to change in color or to release a cleavage product that provides a different color from the original substrate.

The term “fluorescent detectable moiety” or “fluorophore” as used herein refers to a molecule that absorbs light at a particular wavelength (excitation frequency) and subsequently emits light of a longer wavelength (emission frequency). A fluorescent detectable moiety can be stimulated by a laser with the emitted light captured by a detector. The detector can be a charge-coupled device (CCD) or a confocal microscope, which records its intensity.

The term “array” as used herein refers to a two-dimensional spatial grouping or an arrangement. In some embodiments, an array refers to a two-dimensional grouping of oligonucleotides (e.g. primers or capture sequences), which serve to interrogate mixtures of target molecules administered to the surface of the array.

The term “capture oligonucleotide” or “capture sequence” as used herein refers to an oligonucleotide having a recognition sequence and coupled to a solid surface to hybridize with an oligonucleotide having a “tagging sequence” complementary to the recognition sequence, thereby capturing the target oligonucleotide on the solid surface.

The term “solid support” refers to a material having a rigid or semi-rigid surface or surfaces. In certain embodiments, the solid support will take the form of beads, resins, gels, microspheres, films, matrix layers, silica, or other configurations. In some embodiments, at least one surface of the solid support will be substantially flat.

The term “dosage analysis” as used herein describes an assay by which deletions, duplications, and/or rearrangements in the CYP2A6 gene are detected. The peak height, in relative fluorescent intensity, for each fragment amplified from the CYP2A6 gene is compared to one or more internal controls. The term “deletion” as used herein encompasses a mutation that removes one or more nucleotides from the nucleic acid. Conversely, the term “duplication” refers to a mutation that inserts one or more nucleotides of identical sequence directly next to this sequence in the nucleic acid. In a preferred embodiment, a deletion or duplication involves a segment of four or more nucleotides.

A substantial decrease in the amount of a CYP2A6 fragment identified means that the fragment has been deleted and/or converted while a substantial increase in the amount of a CYP2A6 fragment identified means that the fragment has been duplicated The term “substantial decrease” or “substantial increase” means a decrease or increase of at least about 30-50%. Thus, deletion of a single CYP2A6 exon would appear in the assay as a signal representing for example about 50% of the same exon signal from an identically processed sample from an individual with a wildtype CYP2A6 gene. Conversely, amplification of a single exon would appear in the assay as a signal representing for example about 150% of the same exon signal from an identically processed sample from an individual with a wildtype CYP2A6 gene.

As used herein, “about” means in quantitative terms plus or minus 10% unless otherwise indicated.

Units, prefixes, and symbols are denoted in their accepted SI form. Unless otherwise indicated, nucleic acids are written left to right in 5′ to 3′ orientation; amino acid sequences are written left to right in amino to carboxy orientation. Amino acids are referred to herein by either their commonly known three letter symbols or by the one-letter symbols recommended by the IUPAC-IUBMB Nomenclature Commission. Nucleotides, likewise, are referred to by their commonly accepted single-letter codes.

### BRIEF DESCRIPTION OF THE DRAWINGS

FIGS. 1A, 1B, 1C, and 1D show the nucleotide sequence of the Homo Sapiens Cytochrome P450, CYP2A6 gene provided at GenBank Accession No. EU135979 (SEQ ID NO: 60).

### DETAILED DESCRIPTION OF THE PREFERRED EMBODIMENTS

The invention is drawn to assays for the detection of mutations, deletion, duplication, and/or rearrangement in the CYP2A6 gene.

In one aspect, methods are provided to amplify the CYP2A6 gene in a multiplex PCR followed by single nucleotide primer extension to detect 10 missense mutations using fluorescently labeled ddNTPs. The methods provide a primer set capable of correctly genotyping the missense mutations without amplification of the CYP2A7 gene or pseudogenes of CYP2A6 and CYP2A7.

In another aspect, methods are provided for dosage analysis of specific mutations of CYP2A6 using multiplex PCR and fluorescently labeled oligonucleotides. Dosage analysis is used to detect gene deletion, duplication, and/or rearrangement.

Sample Preparation

Specimens from which target nucleic acids can be detected and quantified are from sterile and/or non-sterile sites. Sterile sites from which specimens can be taken are body fluids such as blood (whole blood, serum, plasma), urine, cerebrospinal fluid (CSF), synovial fluid, pleural fluid, pericardial fluid, intraocular fluid, tissue biopsies or endotracheal aspirates. Non-sterile sites from which specimens can be taken are e.g., sputum, stool, swabs from e.g., skin, inguinal, nasal, pharyngeal and/or throat. In certain preferred embodiments, the specimen is blood.

The sample can be processed to release or otherwise make available a nucleic acid for detection as described herein, Such processing can include steps of nucleic acid manipulation, e.g., preparing a cDNA by reverse transcription of RNA from the sample. Thus, the nucleic acid to be amplified by the methods of the invention can be genomic DNA, cDNA, single stranded DNA or mRNA.

The nucleic acid (DNA and/or RNA) can be isolated from the sample according to any methods well known to those of skill in the art. If necessary, the sample can be collected or concentrated by centrifugation and the like. The cells of the sample can be subjected to lysis, such as by treatment with enzymes, heat surfactants, ultrasonication or a combination thereof. The lysis treatment is performed in order to obtain a sufficient amount of CYP2A6 DNA, if present in the sample, to detect using polymerase chain reaction.

Various methods of DNA extraction are suitable for isolating the DNA. Suitable methods include phenol and chloroform extraction. See Maniatis et al., Molecular Cloning, A Laboratory Manual, 2d, Cold Spring Harbor Laboratory Press, page 16.54 (1989). Numerous commercial kits also yield suitable DNA including, but not limited to, QIAamp™ mini blood kit, Agencourt Genfind™, Roche Cobas® Roche MagNA Pure® or phenol:chloroform extraction using Eppendorf Phase Lock Gels®. The nucleic acid isolation techniques and protocols described herein can be used to isolate nucleic acid from a variety of patient samples or sources.

Genomic DNA or cDNA can be subject to amplification by the polymerase chain reaction or related methods using primers directed to specific portions of the CYP2A6 gene which contain a mutation to be detected. In preferred embodiments, genomic DNA is amplified.

A target nucleic acid can be a polymorphic region of a chromosomal nucleic acid, for example, a gene, or a region of a gene potentially having a mutation. Target nucleic acids include, but are not limited to, nucleotide sequence motifs or patterns specific to a particular disease and causative thereof, and to nucleotide sequences specific as a marker of a disease but not necessarily causative of the disease or condition. For example, target nucleic acids can include disease marker genes (including DNA and mRNA corresponding to the disease marker gene), single nucleotide polymorphisms, and microorganisms (i.e. bacteria and viruses). A target nucleic acid also can be a nucleotide sequence that is of interest for research purposes, but that may not have a direct connection to a disease or that may be associated with a disease or condition, although not yet proven so.

Primers and Primer Landing Sites (Amplification and Extension Primers)

Table 1 shows regions of the CYP2A6 gene suitable for amplification using primers capable of specifically amplifying fragments of the CYP2A6 gene without amplification of fragments of the CYP2A7 gene, or pseudogenes of CYP2A6 and CYP2A7. The length of the amplification primers for use in the methods depends on several factors including the nucleotide sequence identity and the temperature at which these nucleic acids are hybridized or used during in vitro nucleic acid amplification.

Primers that amplify a nucleic acid molecule can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection (e.g., by electrophoresis or real-time PCR), similar melting temperatures for the members of a pair of primers, and the length of each primer (i.e., the primers need to be long enough to anneal with sequence-specificity and to initiate synthesis but not so long that fidelity is reduced during oligonucleotide synthesis). In certain preferred embodiments, primers are 12 to 35 nucleotides in length.

A mix of primers can exhibit degeneracy at one or more nucleotide positions. Degenerate primers are used in PCR where variability exists in the target sequence, i.e. the sequence information is ambiguous. Typically, degenerate primers will exhibit variability at no more than about 4, no more than about 3, preferably no more than about 2, and most preferably, no more than about 1 nucleotide position.

Provided herein are the sequences of primers suitable for PCR amplification of portions of the CYP2A6 gene which contain CYP2A6 mutations, using genomic DNA as the template (see, for example, Table 2 and Table 3 below). Primers specifically amplify the CYP2A6 gene but not the CYP2A7 gene nor the pseudogenes of CYP2A6 and CYP2A7.

In one aspect, the invention relates to one or more substantially purified primers, designed to amplify regions of the CYP2A6 gene, having sequences selected from the sequences shown in Table 2 and Table 3. The letter F or R at the end of the primer name indicates whether the primer is a forward (F) or reverse (R)PCR primer. FAM refers to fluorescent compounds chemically linked to the 5′ end of the primer.

The methods described herein provide extension primers that are useful for detecting the CYP2A6 mutations. Accordingly, provided are substantially purified nucleic acids comprising 15-30 nucleotides complementary to a segment of the CYP2A6 gene that is upstream of a mutation site and terminating one nucleotide short of that mutation site. CYP2A6 extension primers can be labeled with a tag or member of a binding pair. Exemplary extension primers are provided in Table 3.

Preferably, an extension primer has a nucleotide sequence that hybridizes in a complementary fashion to a portion of the CYP2A6 gene immediately upstream of a mutation such that the hybridized extension primer terminates one nucleotide 5′ to the mutation site. Accordingly, extension of that primer by one base will incorporate the nucleotide in the mutation site. One of skill in the art would recognize that the extension primer could be designed to be either the sense or anti-sense strand of DNA; in either case, the extension primer would be designed so that when that primer is extended through incorporation of a nucleotide, the nucleotide incorporated corresponds to the mutation site. Extension primers should be of a length sufficient to provide specific hybridization to the target sequence of interest. Such primers preferably comprise an exact complement to the sequence of interest for 12 to 55 nucleotides in length, preferably 15 to 40 nucleotides in length, and more preferably from 15 to 30 nucleotides in length. The extension primer sequence has a 3′ terminus that pairs with a nucleotide base that is, in the sample nucleic acid to which the primer is hybridized, one base 5′ to the mutation site. Suitable extension primers are described herein, and can be one of the sequences set forth in SEQ ID NOs: 41-59.

In addition to the sequence that ensures hybridization to the target site, an extension primer can have additional nucleotides added to the 5′ end that need not participate in specific binding to the CYP2A6 sequence. Thus, such primers can extend for 4 to 50 nucleotides in length, preferably 4 to 40 nucleotides in length, and more preferably from 4 to 30 nucleotides in length, beyond the CYP2A6 sequence. The extension products can be detected both by size and by fluorescent label. In some embodiments, the additional 5′ sequence can include a member of a binding pair such as an oligonucleotide tag. Such an oligonucleotide tag can be complementary to an oligonucleotide conjugated to the surface of a bead. In this embodiment, the extension primer can be captured by hybridization of the oligonucleotide tag on the extension primer to the complementary oligonucleotide on the bead. Exemplary extension primers comprising an oligonucleotide tag are provided in Table 3 with the oligonucleotide tag portion of the primer shown in bold.

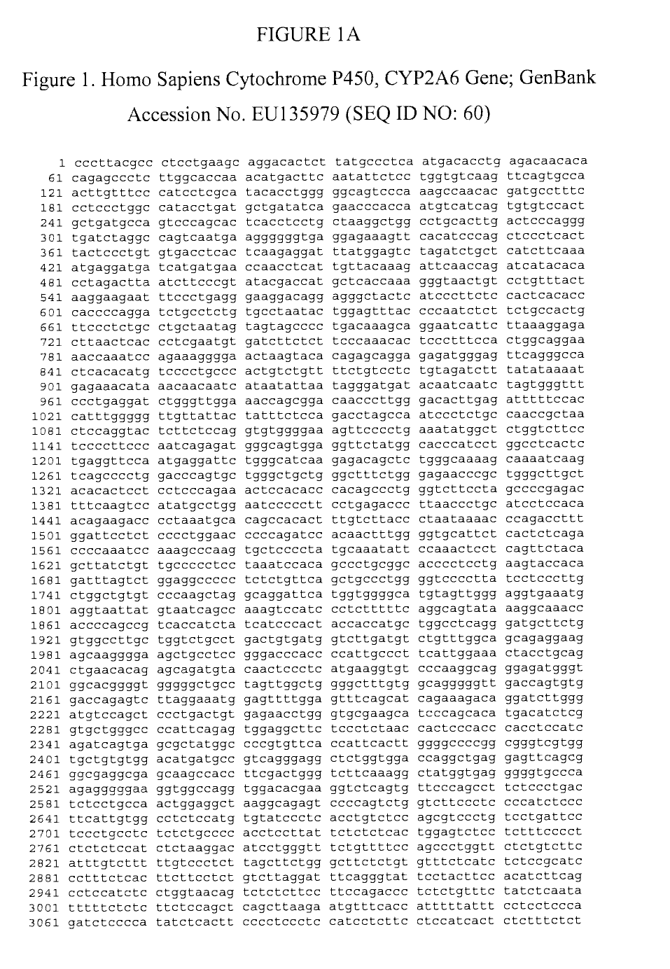

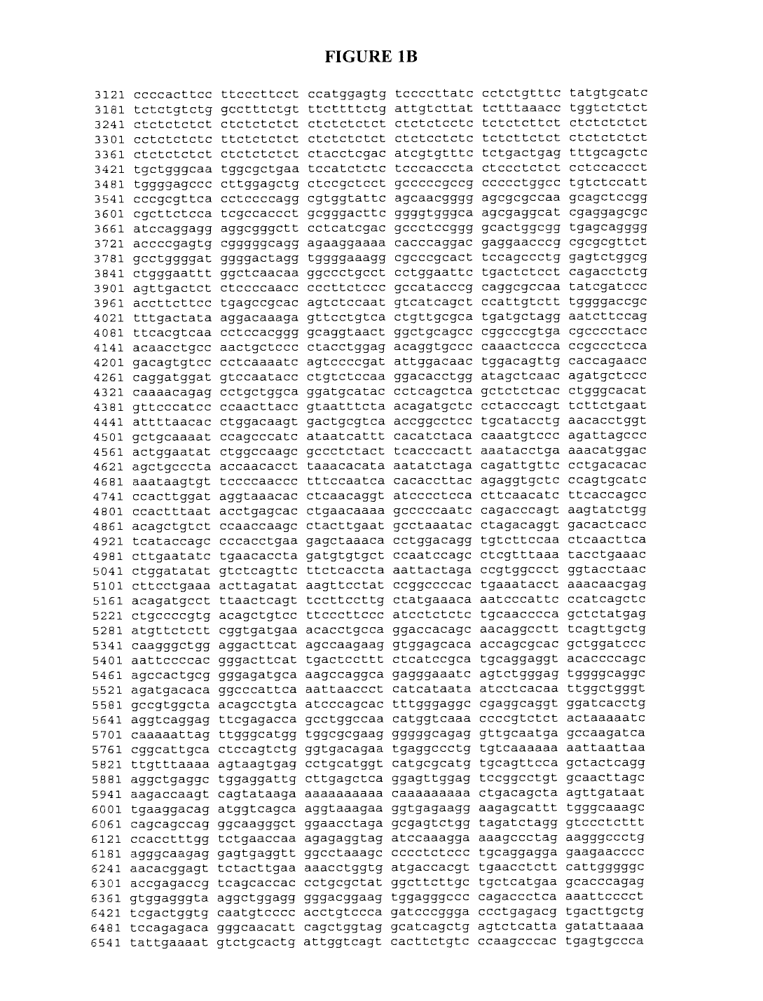

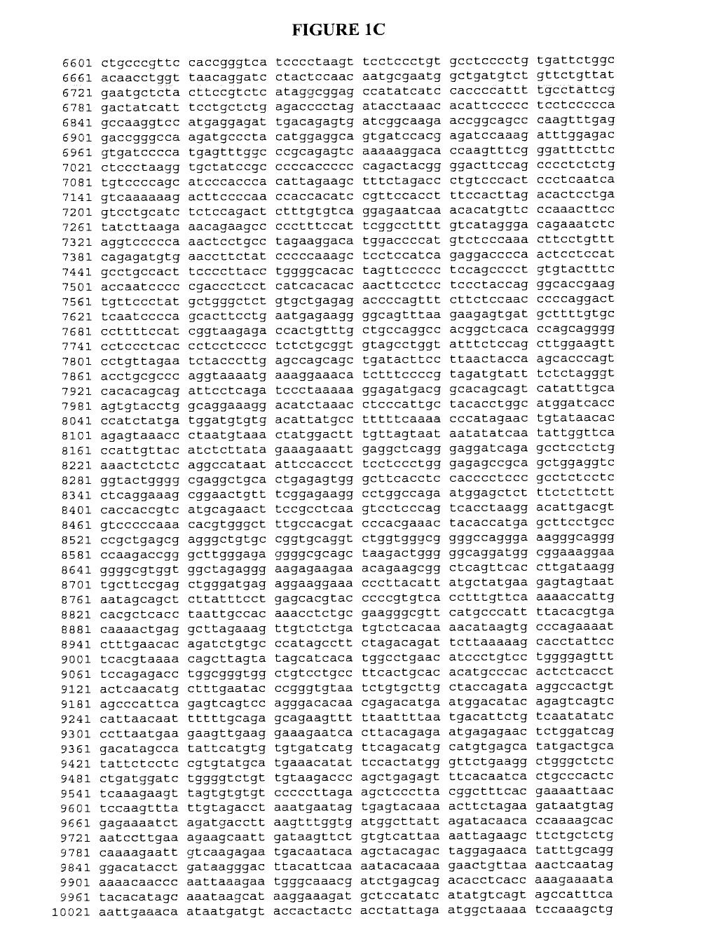

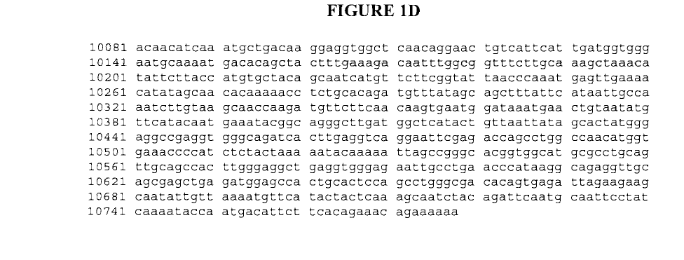

TABLE 1
Suitable Regions for Oligonucleotide Hybridization

| | | |
|---|---|---|
| | | Position of Primer |
| | | Annealing in GenBank |
| SEQ | | Accession No. EU135979 |
| ID | | (FIG. 1): Primer location |
| NO: | Sequence | in CYP2A6 Gene |

| | | |
|---|---|---|
| 1 | 5′-GTCACGTGTAAAATGGGCATGAACGCCCTTCGCA-3′ | 8848-8881: exon 9 |
| 2 | 5′-GCAGTCATATTTGCAAGTGTACCTGGCAGGAAAGGACAT-3′ | 7966-8004: exon 9 |
| 3 | 5′-GGATTGAAGTCCTGGGGGTTGGAGAAGAAACTGGGG-3′ | 7592-7627: exons 7 and part |
| | | of 8 |
| 4 | 5′-AGGCGGAGCCATATCATCCACCCCATTTTGCCTATT-3′ | 6743-6778: exons 7 and part |
| | | of 8 |
| 5 | 5′-GTCATCTGCCTGCCCCACTCCCAGACTGATT-3′ | 5497-5527: exon 5 |
| 6 | 5′-CAGGATGGATGTCCAATACCCTGTCTCCAAGGACACC-3′ | 4261-4297: exon 5 |
| 7 | 5′-ACATCCATCCTGGGTTCTGGTGCAACTGTCCAGTTG-3′ | 4237-4272: exons 3 and 4 |
| 8 | 5′-CCGCCGCCCCCTGGCCTGTCTCCATTCCCGCG-3′ | 3515-3546: exons 3 and 4 |
| 9 | 5′-GGAGCTGGACATCCCAAGATCCTGTCTTTCTGATGCTG-3′ | 2195-2232: exon 1 |
| 10 | 5′-GAAGACCCCTAAATGCACAGCCACACTTTGTCTTAC-3′ | 1444-1479: exon 1 |

TABLE 2
Primers with 5′ Optional Sequence

| | | |
|---|---|---|
| | | Position of Primer Annealing in |
| SEQ | | GenBank Accession No. |
| ID | Sequence with 5′ Optional Sequence | EU 135979 (FIG. 1): Primer |
| NO: | shown in bold and underlined | location in CYP2A6 Gene |

| | | |
|---|---|---|
| 11 | 5′-GTCACGTGTAAAATGGGCATGAACGCCC-3′ | 8848-8875: exon 9 |
| 12 | 5′-GCAGTCATATTTGCAAGTGTACCTGGCAGGAAA-3′ | 7966-7998: exon 9 |
| 13 | 5′-GGATTGAAGTCCTGGGGGTTGGAGAAGAAA-3′ | 7592-7621: exons 7 and part of 8 |
| 14 | 5′-AGGCGGAGCCATATCATCCACCCCATTTTG-3′ | 6743-6772: exons 7 and part of 8 |
| 15 | 5′-GTCATCTGCCTGCCCCACTCCCAGA-3′ | 5497-5521: exon 5 |
| 16 | 5′-CAGGATGGATGTCCAATACCCTGTCTCCAAG-3′ | 4261-4291: exon 5 |
| 17 | 5′-ACATCCATCCTGGGTTCTGGTGCAACTGTC-3′ | 4237-4266: exons 3 and 4 |
| 18 | 5′-CCGCCGCCCCCTGGCCTGTCTCCATT-3′ | 3515-3540: exons 3 and 4 |
| 19 | 5′-GGAGCTGGACATCCCAAGATCCTGTCTTTCTG-3′ | 2195-2226: exon 1 |
| 20 | 5′-GAAGACCCCTAAATGCACAGCCACACTTTG-3′ | 1444-1473: exon 1 |

TABLE 3
Primers

| | | | |
|---|---|---|---|
| | | | Position of Primer |
| | | | Annealing in |
| | | | Gen Bank Accession |
| | | | No. EU135979 (FIG. |
| SEQ | | | 1): Primer location |
| ID | | | in the CYP2A6 Gene |
| NO: | Primer Name | Sequence | (Mutation Detected) |

PCR Primers and Control Primers

| | | | |
|---|---|---|---|
| 21 | 2A6UTR-AS1 | 5′-TGTAAAATGGGCATGAACGCCC-3′ | 8854-8875: exon 9 |
| 22 | 2A6E9R3 | 5′-ATATTTGCAAGTGTACCTGGCAGGAAA-3′ | 7972-7998: exon 9 |
| 23 | 2A6E78F3 | 5′-AAGTCCTGGGGGTTGGAGAAGAAA-3′ | 7598-7621: exons 7 |
| | | | and part of 8 |
| 24 | 2A6E78R2 | 5′-AGCCATATCATCCACCCCATTTTG-3′ | 6749-6772: exons 7 |
| | | | and part of 8 |
| 25 | 2A6E5F2 | 5′-TGCCTGCCCCACTCCCAGA-3′ | 5503-5521: exon 5 |
| 26 | 2A6E5R2 | 5′-GGATGTCCAATACCCTGTCTCCAAG-3′ | 4267-4291: exon 5 |
| 27 | 2A6E34F2 | 5′-ATCCTGGGTTCTGGTGCAACTGTC-3′ | 4243-4266: exons 3 |
| | | | and 4 |
| 28 | 2A6E34R2 | 5′-CCCCCTGGCCTGTCTCCATT-3′ | 3521-3540: exons 3 |
| | | | and 4 |
| 29 | 2A6E1F2 | 5′-GGACATCCCAAGATCCTGTCTTTCTG-3′ | 2201-2226: exon 1 |
| 30 | 2A6E1R2 | 5′-CCCTAAATGCACAGCCACACTTTG-3′ | 1450-1473: exon 1 |
| 31 | 2A7Fben-FAM | 5′-6-FAM/CTTTGGATTCCTCTCCCTTGGAATG-3′ | N/A |
| 32 | 2A7Rben | 5′-GGGACACCTTCATGATGGAGTCAC-3′ | N/A |
| 33 | 1B1SNPEx3F5′- | 5′-6-FAM/CAGTGTATCCTGATGTGCAGACTCGAG-3 | N/A |
| | FAM | | |
| 34 | 1B1SNPEx3R5′ | 5′-CAGTCAGTCAGTTTATTGGCAAGTTTCCTTGGC-3′ | N/A |
| 35 | CFDEL22F- | 5′-6-FAM/GTTGGGCTCAGATCTGTGATAGA-3′ | N/A |
| | FAM | | |
| 36 | CFDEL23R | 5′-CAAGGGCAATGAGATCTTAAGTAA-3 | N/A |
| 37 | 2A6UTR-AS1- | 5′-6-FAM/TGTAAAATGGGCATGAACGCCC-3′ | 8854-8875: exon 9 |
| | FAM | | |
| 38 | 2A6E78F3-FAM | 5′-6-FAM/AAGTCCTGGGGGTTGGAGAAGAAA-3′ | 7598-7621: exons 7 |
| | | | and part of 8 |
| 39 | 2A6E34F2-FAM | 5′FAM/ATCCTGGGTTCTGGTGCAACTGTC-3′ | 4243-4266: exons 3 |
| | | | and 4 |
| 40 | 2A6E1R2-FAM | 5′-FAM/CCCTAAATGCACAGCCACACTTTG-3′ | 1450-1473: exon 1 |

Exemplary Extension Primers

| | | | |
|---|---|---|---|
| 41 | 2A6*5R2 | 5′-CGTGTCCCCCAAACACGTGG-3′ | 8458-8477: exon 9 |
| | | | (*5) |
| 42 | 2A6*2F2 | 5′-ACCGCCAGTGCCCCGG-3′ | 3696-3711: exon 3 |
| | | | (*2) |
| 43 | 2A6*6R2 | 5′-GCGCGCCAAGCAGCTCC-3′ | 3582-3598: exon 3 |
| | | | (*6) |
| 44 | 2A6*7R3 | 5′-CTCAAGTCCTCCCAGTCACCTAAGGACA-3′ | 8426-8453: exon 9 |
| | | | (*7) |
| 45 | 2A6*18R2 | 5′-CTCCCTACCAGGGCACCGAAGTGT-3′ | 7540-7563: exon 8 |
| | | | (*18) |
| 46 | 2A6*8F2 | 5′-GGGCAGGAAGCTCATGGTGTAGTTT-3′ | 8497-8521: exon 9 |
| | | | (*8) |
| 47 | 2A6*9F3 | 5′-GGCTGGGGTGGTTTGCCTTT-3′ | 1850-1869: before |
| | | | exon 1 (*9) |
| 48 | 2A6*11F3 | 5′-TGGCAGGTGTTTCATCACCGAAG-3′ | 5288-5310: exon 5 |
| | | | (*11) |
| 49 | 2A6*17R2 | 5′-TCCACGAGATCCAAAGATTTGGAGAC-3′ | 6935-6960: exon 7 |
| | | | (*17) |
| 50 | 2A6*20R2 | 5′TGGGGACCGCTTTGACTATAAGGACA-3′ | 4011-4036: exon 4 |
| | | | (*20) |

Exemplary Extension Primers Containing an
Oligonucleotide Tag shown in bold

| | | | |
|---|---|---|---|
| 51 | 2A6*2F2 | 5′-TTTTACCGCCAGTGCCCCGG-3′ | 3696-3711: exon 3 |
| | | | (*2) |
| 52 | 2A6*6R2 | 5′-TGACTGACTGACTGCGCGCCAAGCAGCTCC-3′ | 3582-3598: exon 3 |
| | | | (*6) |
| 53 | 2A6*7R3 | 5′-CTGACTGACTCAAGTCCTCCCAGTCACCTAAGGACA- | 8426-8453: exon 9 |
| | | 3′ | (*7) |
| 54 | 2A6*18R2 | 5′-CTGACTGACTCCCTACCAGGGCACCGAAGTGT-3′ | 7540-7563: exon 8 |
| | | | (*18) |
| 55 | 2A6*8F2 | 5′-TGACTGACTGACTGGGCAGGAAGCTCATGGTGTAGT | 8497-8521: exon 9 |
| | | TT-3′ | (*8) |
| 56 | 2A6*9F3 | 5′- | 1850-1869: before |
| | | TGACTGACTGACTGACTGACTGGCTGGGGTGGTTTGC | exon 1 (*9) |
| | | CTTT-3′ | |
| 57 | 2A6*11F3 | 5′- | 5288-5310: exon 5 |
| | | GACTGACTGACTGACTGACTGACTTGGCAGGTGTTTC | (*11) |
| | | ATCACCGAAG-3′ | |
| 58 | 2A6*17R2 | 5′-ACTGACTGACTGACTGACTGACTGACTTCCACGAGA | 6935-6960: exon 7 |
| | | TCCAAAGATTTGGAGAC-3′ | (*17) |
| 59 | 2A6*20R2 | 5′-CTGACTGACTGACTGACTGACTGACTGACTGGGGAC | 4011-4036: exon 4 |
| | | CGCTTTGACTATAAGGACA-3′ | (*20) |

Amplification of Nucleic Acids

Nucleic acid samples or target nucleic acids can be amplified by various methods known to the skilled artisan. Preferably, PCR is used to amplify nucleic acids of interest. Briefly, in PCR, two primer sequences are prepared that are complementary to regions on opposite complementary strands of the marker sequence. An excess of deoxynucleoside triphosphates (dNTPs) are added to a reaction mixture along with a DNA polymerase, e.g., Taq polymerase.

Degraded DNA samples, or DNA samples that are contaminated by inhibitors of PCR, may not be amplified in the PCR reaction. Unidentified DNA polymorphism(s) can affect PCR amplification and/or preclude allelic discrimination. The *2, *4, *5, *6, *7, *8, *9, *11, *12, *17, *18, and *20 alleles in CYP2A6 are detected by the methods provided herein. Additional CYP2A6 alleles may also be detected.

If the target sequence is present in a sample, the primers bind to the sequence and the polymerase causes the primers to be extended along the target sequence by adding on nucleotides. By raising and lowering the temperature of the reaction mixture, the extended primers dissociate from the target nucleic acid to form reaction products. Excess primers bind to the target nucleic acid and to the reaction products and the process is repeated, thereby generating amplification products. Cycling parameters can be varied, depending on the length of the amplification products to be extended. An internal positive amplification control (IC) can be included in the sample. The IC can be used to monitor both the conversion process and any subsequent amplification.

In one preferred embodiment, the target nucleic acids are amplified in a multiplex amplification reaction. A variety of multiplex amplification strategies are known in the art and can be used with the methods described herein. The multiplex amplification strategy can use PCR, RT-PCR or a combination thereof depending on the type of nucleic acid contained in the sample. For example, if an RNA genome is present, RT-PCR can be utilized. The PCR enzyme can be an enzyme with both a reverse transcription and polymerase function. Furthermore, the PCR enzyme may be capable of “hot start” reactions as is known in the art.

In one preferred embodiment, once amplified, the PCR products are treated, e.g., with calf intestinal phosphatase (CIP) and exonuclease I, to remove excess dNTPs and PCR primers, respectively, prior to nucleotide primer extension.

Nucleotide Primer Extension

Nucleotide primer extension is a two step process that first involves the hybridization of a primer to the bases immediately upstream of the SNP nucleotide followed by a ‘mini-sequencing’ reaction, in which DNA polymerase extends the hybridized primer by adding a base that is complementary to the SNP nucleotide. This incorporated base is detected and determines the SNP allele. Because, primer extension is based on the highly accurate DNA polymerase enzyme, the method is generally very reliable. Primer extension is able to genotype most SNPs under very similar reaction conditions making it also highly flexible. The primer extension method is used in a number of assay formats.

Generally, there are two main approaches which use the incorporation of either fluorescently labeled dideoxynucleotides (ddNTP) or fluorescently labeled deoxynucleotides (dNTP). With ddNTPs, primers hybridize to the target DNA immediately upstream of SNP nucleotide, and a single, ddNTP complementary to the SNP allele is added to the 3′ end of the primer (the missing 3′-hydroxyl in dideoxynucleotide prevents further nucleotides from being added). Each ddNTP is labeled with a different fluorescent signal allowing for the detection of all four alleles in the same reaction. With dNTPs, allele-specific primers have 3′ bases which are complementary to each of the SNP alleles being interrogated. If the target DNA contains an allele complementary to the primer's 3′ base, the target DNA will completely hybridize to the primer, allowing DNA polymerase to extend from the 3′ end of the primer. This is detected by the incorporation of the fluorescently labeled dNTPs onto the end of the primer. If the target DNA does not contain an allele complementary to the primer's 3′ base, the target DNA will produce a mismatch at the 3′ end of the primer and DNA polymerase will not be able to extend from the 3′ end of the primer. The benefit of the second approach is that several labeled dNTPs can get incorporated into the growing strand, allowing for increased signal. However, DNA polymerase in some rare cases, can extend from mismatched 3′ primers giving a false positive result. The flexibility and specificity of primer extension make it amenable to high throughput analysis.

In single nucleotide primer extension, a target sequence is amplified (e.g., by PCR) from a sample of nucleic acids. The amplicon is denatured and contacted with a plurality of “SNaPshot” primers. The primers are complementary to the target sequence and are designed to “walk” down the target base-by-base, with a primer provided for each position to be sequenced. The primer is allowed to hybridize to the target in the presence of four differentially labeled ddNTPs. A polymerase catalyzes a single-base primer extension reaction. Thus, the sequence of the nucleotide position adjacent to the 3′ end of the primer sequence is determined by the particular label of the incorporated dideoxy nucleotide.

Typically, one Snapshot primer is used per nucleotide to be sequenced. However, where the region of the target corresponding to the SNaPshot primer contains known polymorphisms (particularly at the 3′ end of the primer), multiple SNaPshot primers must be generated. This ensures that at least one primer is complementary to the target, allowing for primer extension. Where the region of the target corresponding to the SNaPshot primer contains unknown polymorphisms, and the SNaPshot primer fails to hybridize, no primer extension will occur (or it may occur at significantly reduced levels). If there is no detectable ddNTP incorporation, then the system will read that position as a deletion.

The single nucleotide primer extension reaction can be performed in a multiplex format and the detection step can be adjusted by use of, for example, distinguishably labeled ddNTPs, distinguishably tagged extension primers (allowing for capture of the extension primers on distinguishably labeled beads), and multiple lasers and detectors (allowing for the simultaneous detection of multiple labels) so that the skilled artisan can determine the identity of the nucleotide added to each primer. In other embodiments of this multiplex single nucleotide primer extension reaction, labeled extension primers can be dissociated from the amplicons (by for example, heat denaturation) following the extension reaction and separated from the amplicons by, for example, degradation of the amplicons, or blocking of the re-hybridization of the labeled extension primers to the amplicons with the addition of excess unlabeled extension primers.

Specifically, in single nucleotide primer extension, an oligonucleotide primer is designed to have a 3′ end that is one nucleotide 5′ to a specific mutation site. In some embodiments, the extension primers are labeled with a tag or a member of a binding pair to allow the capture of the primer on solid phase. In particular embodiments, the primers can be tagged with varying lengths of nonspecific polynucleotides (e.g., poly-GACT) to allow multiplex detection of preferably 2 or more, more preferably 3 or more, 4 or more, 5 or more, even 10 or more different mutations (polymorphisms) in a single reaction. The primer hybridizes to the PCR amplicon in the presence of one or more labeled ddNTPs and a DNA polymerase. The polymerase extends the primer by one nucleotide, adding a single, labeled ddNTP to the 3′ end of the extension primer. The addition of a dideoxy nucleotide terminates chain elongation. If more than one dideoxynucleotide (e.g., ddATP, ddGTP, ddCTP, ddTTP, ddUTP, etc.) is used in a reaction, one or more can be labeled. If multiple labels are used, the labels can be distinguishable e.g., each is labeled with a different fluorescent colored dye. The products are labeled oligonucleotides, each one of which can be detected based on its label.

Detection of Amplified Nucleic Acids

Amplification of nucleic acids can be detected by any of a number of methods well-known in the art such as, for example, end-point detection, capillary electrophoresis, gel electrophoresis, column chromatography, “real-time” detection, solid-support sequencing, hybridization with a probe, or melting curve analysis.

The skilled artisan will understand that, if one wishes to determine if a specific genotype is present in a sample, e.g., a “T” in a certain position in the CYP2A6 sequence that would normally be a “G” in the wild-type sequence, one need only provide a single labeled ddNTP, in this case ddTTP, with an appropriate extension primer. If the “T” mutation is present, the labeled ddTTP will be incorporated into the 3′ end of the extension primer. In contrast, if the wild-type nucleotide is present, no labeled extension primer will be created. Depending on the polymorphisms selected for analysis, from one to four labeled ddNTPs may be required to perform an assay. One can also choose to include all four ddNTPs or at least two relevant ddNTPs (e.g., the ddNTPs corresponding to the nucleotide in the wild-type and mutant sequences) in a reaction for convenience, or so that mutant and wild-type sequences can be labeled.

To assist in identifying amplified segments, at least one primer suitable for amplifying a target fragment can be labeled with a detectable moiety. It would be evident to the skilled artisan that the detectable moiety could be attached in any manner of variety that does not interfere with the primer to function as an amplification primer.

End-Point Detection

In end-point detection, the amplicon(s) is detected by first size-separating the amplicons, then detecting the size-separated amplicons. The separation of amplicons of different sizes can be accomplished by, for example, gel electrophoresis, column chromatography, or capillary electrophoresis. These and other separation methods are well-known in the art. The separated nucleic acids can then be stained with a dye such as ethidium bromide and the size of the resulting stained band or bands can be compared to a standard DNA ladder.

Electrophoresis

Dideoxynucleotides are useful in the sequencing of DNA in combination with electrophoresis. A DNA sample which undergoes PCR (polymerase chain reaction) in a mixture containing all four deoxynucleotides and one dideoxynucleotide will produce strands of length equal to the position of each base of the type that complements the type that has a dideoxynucleotide present. That is, each nucleotide base of that particular type has a probability of being bonded to not a deoxynucleotide, but rather a dideoxynucleotide, which ends chain elongation. Thus, if the sample then undergoes electrophoresis, there will be a band present for each length at which the complement of the dideoxynucleotide is present. It is now common to use fluorescent dideoxynucleotides such that each one of the four has a different fluorescence that can be detected by a sequencer, thus only one reaction is needed.

Amplified target segments can be efficiently evaluated by size (determined by electrophoretic mobility) and/or detectable moiety using commercially available automated systems. For example, ABI PRISM® 3100 Genetic Analyzer can be used with an ABI PRISM 3100 capillary array, 36-cm (P/N#4315931). This provides a multi-color fluorescence-based DNA analysis system that uses capillary electrophoresis (CE) with 16 capillaries operating in parallel to separate labeled PCR products. A CE DNA sequencer/analyzer that operates 96 capillaries may be preferable in assays in which 96-well plates are used. Analyzers with the capacity to process 96 wells include the MegaBACE™ 1000 DNA Analysis System (Molecular Dynamics, Inc and Amersham Pharmacia Biotech) and the 3700 DNA Analyzer from (Perkin-Elmer Biosystems).

Capillary electrophoresis (CE), or capillary zone electrophoresis (CZE), can be used to separate ionic species by their charge and frictional forces. In traditional electrophoresis, electrically charged analytes move in a conductive liquid medium under the influence of an electric field. The technique of capillary electrophoresis (CE) separates species based on their size to charge ratio in the interior of a small capillary filled with an electrolyte.

Fluorescence detection can also be used in capillary electrophoresis for samples that naturally fluoresce or are chemically modified to contain fluorescent tags. This mode of detection offers high sensitivity and improved selectivity for these samples, but cannot be utilized for samples that do not fluoresce.

Real-Time Detection

In one approach, sequences from two or more fragments of interest are amplified in the same reaction vessel (i.e. “multiplex PCR”). Detection can take place by measuring the end-point of the reaction or in “real time.” For real-time detection, primers and/or probes can be detectably labeled to allow differences in fluorescence when the primers become incorporated or when the probes are hybridized, for example, and amplified in an instrument capable of monitoring the change in fluorescence during the reaction. Real-time detection methods for nucleic acid amplification are well known and include, for example, the TaqMan® system, the Scorpion bi-functional molecule, and the use of intercalating dyes for double stranded nucleic acid.

In a suitable embodiment, real time PCR is performed using any suitable instrument capable of detecting fluorescence from one or more fluorescent labels. For example, real time detection on the instrument (e.g., a ABI Prism® 7900HT sequence detector) monitors fluorescence and calculates the measure of reporter signal, or Rn value, during each PCR cycle. The threshold cycle, or Ct value, is the cycle at which fluorescence intersects the threshold value. The threshold value is determined by the sequence detection system software or manually.

Solid Support Sequencing

Detection of the labeled extension primer can include capture of the extension primer on a bead (or other solid phase) and subjecting the bead to analysis, such as by flow cytometry, to detect the labeled primer bound to the bead. Preferably, the primer comprises one member of a “binding pair,” which refers herein to two molecules which form a complex through a specific interaction. Thus, the extension primer can be captured on a bead through an interaction between one member of the binding pair linked to the extension primer and the other member of the binding pair coupled to the bead. In one embodiment, one member of the binding pair is an oligonucleotide sequence which is part of the extension primer, and the other member of the binding pair is the complement of that binding pair oligonucleotide sequence, which is coupled to a bead. In other embodiments the binding pair is comprised of a ligand-receptor, a hormone-receptor, or an antigen-antibody.

In certain embodiments, a binding pair can be employed in the capture of the extension primer on solid phase and also in the detection of the added nucleotide. In these embodiments, it is desirable that a different binding pair be used in each system (i.e., extension primer capture and detection of added nucleotide) in order to avoid competing binding between the two systems.

A multiplex single nucleotide primer extension reaction can be performed using extension primers corresponding to the site of each mutation and distinguishably labeled ddNTPs. The identity of the nucleotide added to the extension primer is determined by detection of the label incorporated into the extension primer through the addition of a labeled ddNTP. In certain embodiments, the label can be coupled to the ddNTP through a covalent attachment. In other embodiments, a binding pair member also can be used to link the detectable label to the ddNTP. In one embodiment, each extension primer comprises a distinct oligonucleotide tag, which is complementary to a oligonucleotide on a distinguishably labeled bead. Thus, following the primer extension reaction, the labeled extension primers can be captured on distinguishably labeled beads. Flow cytometry can then be used to separate beads by label and detect and identify any signal contained in the attached extension primer.

In some embodiments, the sequencing of contiguous nucleotides is carried out, at least in part, using a solid support. A variety of different supports can be used. In some embodiments, the solid support is a single solid support, such as a chip or wafer, or the interior or exterior surface of a tube, cone, or other article. In some embodiments, primers can be immobilized at defined positions on the solid support to generate a two dimensional array. The solid support is fabricated from any suitable material to provide an optimal combination of such desired properties as stability, dimensions, shape, and surface smoothness. Preferred materials do not interfere with nucleic acid hybridization and are not subject to high amounts of non-specific binding of nucleic acids. Suitable materials include biological or nonbiological, organic or inorganic materials. For example, an array can be fabricated from any suitable plastic or polymer, silicon, glass, ceramic, or metal, and can be provided in the form of a solid, resin, gel, rigid film, or flexible membrane. Suitable polymers include, for example, polystyrene, poly(alkyl)methacrylate, poly(vinylbenzophenone), polycarbonate, polyethylene, polypropylene, polyamide, polyvinylidenefluoride, and the like. Preferred materials include polystyrene, glass, and silica.

Dimensions of the solid support are determined based upon such factors as the desired number of regions and the number of sequences to be assayed. As an example, a solid support can be provided with planar dimensions of about 0.5 cm to about 7.5 cm in length, and about 0.5 cm to about 7.5 cm in width. Solid supports can also be singly or multiply positioned on other supports, such as microscope slides (e.g., having dimensions of about 7.5 cm by about 2.5 cm). The dimensions of the solid support can be readily adapted for a particular application.

In some embodiments, the solid support is a particulate support, also referred to as a microsphere, bead or particle. In particular embodiments, the particles are conjugated directly to the extension primers. In other embodiments, capture oligonucleotides are coupled to particles. Typically, the particles form groups in which particles within each group have a particular characteristic, such as, for example, color, fluorescence frequency, density, size, or shape, which can be used to distinguish or separate those particles from particles of other groups. Preferably, the particles can be separated using techniques, such as, for example, flow cytometry.

The particles can be fabricated from virtually any insoluble or solid material. For example, the particles can be fabricated from silica gel, glass, nylon, resins, Sephadex™ Sepharose™, cellulose, magnetic material, a metal (e.g., steel, gold, silver, aluminum, copper, or an alloy) or metal-coated material, a plastic material (e.g., polyethylene, polypropylene, polyamide, polyester, polyvinylidenefluoride (PVDF)) and the like, and combinations thereof. Examples of suitable micro-beads are described, for example, in U.S. Pat. Nos. 5,736,330, 6,046,807, and 6,057,107, all of which are incorporated herein by reference.

In certain embodiments, the support (whether a two-dimensional array or particulate support) is capable of binding or otherwise holding an extension primer to the surface of the support in a sufficiently stable manner to accomplish the purposes described herein. Such binding can include, for example, the formation of covalent, ionic, coordinative, hydrogen, or van der Waals bonds between the support and the extension primer or attraction to a positively or negatively charged support. Extension primers are attached to the solid support surface directly or via linkers. In one embodiment, well-known chemical crosslinkers can be used for covalent linkage. For example, amino-labeled primers can be covalently attached to carboxylated solid supports using N-(3-Dimethylaminopropyl)-N′-ethylcarbodiimide hydrochloride (EDAC). In another example, the surface of Luminex™ particles can be modified with, for example, carboxylate, maleimide, or hydrazide functionalities or avidin and glass surfaces can be treated with, for example, silane or aldehyde (to form Schiff base aldehyde-amine couplings with DNA). In some embodiments, the support or a material disposed on the support (as, for example, a coating on the support) includes reactive functional groups that can couple with a reactive functional group on the capture oligonucleotides. As examples, the support can be functionalized (e.g., a metal or polymer surface that is reactively functionalized) or contain functionalities (e.g., a polymer with pending functional groups) to provide sites for coupling the capture oligonucleotides.

As yet another alternative, the support can be partially or completely coated with a binding agent, such as streptavidin, antibody, antigen, enzyme, enzyme cofactor or inhibitor, hormone, or hormone receptor. The binding agent is typically a biological or synthetic molecule that has high affinity for another molecule or macromolecule, through covalent or non-covalent bonding. The extension primer is coupled to a complement of the binding agent (e.g., biotin, antigen, antibody, enzyme cofactor or inhibitor, enzyme, hormone receptor, or hormone). The extension primer is then brought in contact with the binding agent to hold the capture oligonucleotide on the support. Other known coupling techniques can be readily adapted and used in the systems and methods described herein.

In one embodiment, microspheres, which are uniquely distinguished by detectable characteristics, are utilized. The microspheres are alternately termed microparticles, beads, polystyrene beads, microbeads, latex particles, latex beads, fluorescent beads, fluorescent particles, colored particles and colored beads. The microspheres serve as vehicles for molecular reactions. Microspheres for use in flow cytometry can be obtained from manufacturers, such as Luminex Corp. of Austin, Tex. Illustrative microspheres and methods of manufacturing same are, for example, found in U.S. Pat. Nos. 6,268,222 and 6,632,526.

Microspheres can be composed of polystyrene, cellulose, or other appropriate material. In a particular embodiment, microspheres are stained with different amounts of fluorescent dyes. Preferably the dyes have the same or overlapping excitation spectra, but possess distinguishable emission spectra. Fluorescent dyes that can be used in the microspheres include cyanine dyes, with emission wavelengths between 550 nm and 900 nm. These dyes may contain methine groups and their number influences the spectral properties of the dye. The monomethine dyes that are pyridines typically have blue to blue-green fluorescence emission, while quinolines have green to yellow-green fluorescence emission. The trimethine dye analogs are substantially shifted toward red wavelengths, and the pentamethine dyes are shifted even further, often exhibiting infrared fluorescence emission (see for example U.S. Pat. No. 5,760,201). However, any dye that is soluble in an organic solvent can be used.

The classification parameters of each microsphere advantageously includes one, two, three, four, or more standard fluorochromes or fluorescent dyes. The one or more fluorochromes are affixed to or embedded in each microsphere by any standard method, for example, by attachment to the microsphere surface by covalent bonding or adsorption. Alternatively, the dye(s) can be affixed by a copolymerization process, wherein monomers, such as an unsaturated aldehyde or acrylate, are allowed to polymerize in the presence of a fluorescent dye, such as fluoroscein isothiocynate (FITC), in the resulting reaction mixture.

Another method by which one or more dyes are embedded in a microsphere includes adding a subset of microspheres to, for example, an organic solvent to expand the microspheres. An oil-soluble or hydrophobic dye, for example, is subsequently added to the subset of microspheres, thereby penetrating into each microsphere. After incubating the resulting combination, an alcohol or water-based solution, for example, is added to the combination and the organic solvent is removed. The microsphere shrinks, retaining the dye(s) inside. Each fluorochrome in the microsphere optionally serves as an additional or alternative classification parameter.

Each of the microspheres are addressed to a unique primer or linker sequence, allowing the analysis of many nucleotide positions in a single reaction. After a single base primer extension reaction, the particles are supplied to a reader system, which determines the particle IDs to identify the particle types and also detects the reporter signals. The reader system includes multiple excitation light sources, such as laser or other devices with controlled wavelengths and optical power, such as LEDs, SLDs, broadband sources with excitation filters, and so forth. The light sources excite the various reporters to supply associated signals to one or more detectors. Emission filters and wavelength discriminators are included such that a given detector receives at a given time the signals associated with a single assay binding label.

In one embodiment, the extension primers are indirectly immobilized to a solid support (e.g. microsphere or two-dimensional array) via a linker sequence. In this embodiment, each extension primer can have a unique nucleic acid 5′ tag sequence, which is complementary to a capture oligonucleotide conjugated to the solid support (e.g., microsphere or two-dimensional array). Thus, the capture oligonucleotide includes a recognition sequence that can capture, by hybridization, a target oligonucleotide having a complementary tagging sequence. The hybridization of the recognition sequence of a capture oligonucleotide and the tagging sequence of a target oligonucleotide results in the coupling of the target oligonucleotide to the solid support. The recognition sequence and tagging sequence are associated with a particular extension primer sequence (also part of the target nucleic acid).

The coding and tagging sequences typically include at least six nucleotides and, in some instances, include at least 8, 10, 15, or 20 or more nucleotides. The capture oligonucleotide also typically includes a functional group that permits binding of the capture oligonucleotide to the solid support or functional groups disposed on or extending from the solid support. The functional group can be attached directly to the polymeric backbone or can be attached to a base in the nucleotidic sequence. As an alternative, the capture oligonucleotide can include a crosslinking portion to facilitate crosslinking, as described above, or can be electrostatically held on the surface. The capture oligonucleotides can be formed by a variety of techniques, including, for example, solid state synthesis, DNA replication, reverse transcription, restriction digest, run-off transcription, and the like. Commercial capture and linker sequence sets are provided by TagIt™ (Luminex, Austin, Tex.) and ZipCode™ (Celera, Rockville, Md.).

In one embodiment, solid supports with associated capture oligonucleotides are disposed in a holder, such as, for example, a vial, tube, or well. After a primer extension reaction, the extension primers are added to the holder under hybridization conditions. The groups of supports are then investigated to determine which support(s) have attached target oligonucleotides. Optionally, the supports can be washed to reduce the effects of cross-hybridization. One or more washes can be performed at the same or different levels of stringency, as described below. As another optional alternative, prior to contact with the support(s) and capture oligonucleotides, the solution containing target oligonucleotides can be subjected to, for example, size exclusion chromatography, differential precipitation, spin columns, or filter columns to remove primers that have not been amplified or to remove other materials that are not the same size as the target oligonucleotides.

In some embodiments, the methods utilize encoded particles having a particular detectable signature that are conjugated to a specific primer, or in the case of a sandwich assay, with a capture oligonucleotide, to form particle types. The sets of particle types are then pooled, and aliquots of the particle types are removed to assay vessels. Samples with at least one or more labeled reporter molecules (one for each nucleotide base A, C, T, and G) are supplied to the respective vessels. Following primer extension, the encoded particles and reporter molecules can be detected using a flow cytometer that is capable of reading the molecule to determine both the identity of the microsphere and of the labeled terminator. Alternatively, primer extension can occur prior to contacting the primers with the encoded particles. A computer can be used to associate the particle ID signature and the reporter molecule with a specific nucleotide base at a particular position.

For each microsphere product supplied, the reader system determines the particle ID and the identity of the reporter (terminator label). Each particle ID is associated with an extension primer designed to interrogate a specific nucleotide position. Using this information, together with data on which label is incorporated into the extension product, the nucleotide base identity can be determined for a specific position on an oligonucleotide template.

Typically, one primer is used per nucleotide to be sequenced. These primers can be provided together in a multiplex reaction, or in separate reaction vessels. Where the region of the target corresponding to the primer contains known polymorphisms (particularly at the 3′ end of the primer), multiple primers can be used to account for the variability. This ensures that at least one primer in the mixture is complementary to the target, allowing for primer extension. Where the region of the target corresponding to the primer contains unknown polymorphisms, the extension primer can fail to anneal to the template and/or provide a 3′hydroxyl for primer extension. Consequently, no primer extension will occur (or it may occur at reduced levels). If there is no detectable terminator incorporation, then the system will read that position as a deletion. A microsphere that produces no primer extension or label incorporation suggests that the extension primer failed to appropriately anneal to the target. Such a result can be indicative of an previously unknown sequence polymorphism within the region of the primer.

In one embodiment of the invention, the solid support is a two-dimensional microarray or biochip. The extension primers are immobilized on the array at predetermined positions either before or after primer extension. In embodiments where the primers are conjugated directly to the array, the primers are extended on the array. In embodiments where the primers are immobilized via a linker sequence, the primers are extended before, after, or during hybridization to the array. Following primer extension, the fluorescent label from the dideoxynucleotide can be detected at a particular position on the array by scanning the fluorescence at each position, thereby allowing for the determination of the sequence at that location.

In one embodiment, the microarray comprises a film-based microarray such as the BioFilmChip™ available from AutoGenomics (Carlsbad, Calif.). These biochips comprise a matrix layer coupled to a substrate, wherein the matrix layer includes a plurality of oligonucleotides in a plurality of predetermined positions. The term “predetermined position” of an analyte refers to a particular position of the analyte on the chip that is addressable by at least two coordinates relative to a registration marker on the chip, and particularly excludes a substantially complete coating of the chip with the analyte and/or probe. Therefore, preferred pluralities of predetermined positions will include an array with a multiple rows of substrates forming multiple columns.

In some embodiments, matrix layers can be multi-functional matrix layers that reduce autofluorescence, incident-light-absorption, charge-effects, and/or surface unevenness of the substrate, and contemplated biochips can comprise additional matrix layers. This microarray can be used with a platform such as the Infiniti™ Analyzer, also available from AutoGenomics (Carlsbad, Calif.). Other suitable approaches include the microarray technology commercially available from a variety of sources.

For each address on the array, the reader system determines the identity of the reporter, i.e. terminator label. Each address is associated with an extension primer designed to interrogate a specific nucleotide position. Using this information, together with data on which label is incorporated into the extension product, the nucleotide base identity can be determined for a specific position on an oligonucleotide template.

Flow Cytometry

Flow cytometry is a technique well-known in the art. Flow cytometers hydrodynamically focus a liquid suspension of particles (e.g., synthetic microparticles, microspheres, or beads) into an essentially single-file stream such that each particle can be analyzed individually. Flow cytometers are capable of measuring forward and side light scattering which correlates with the size of the particle and the particles may have their own label. Thus, particles of differing sizes can be used in invention methods simultaneously to detect distinct nucleic acid segments. In addition fluorescence at one or more wavelengths can be measured simultaneously. Consequently, particles can be sorted by size and/or fluorescence and the fluorescence of one or more associated fluorescently labeled probes can be analyzed. Exemplary flow cytometers include the Becton-Dickenson Immunocytometry Systems FACSCAN.

In one embodiment, flow cytometry is used to analyze the reaction product of the primer extension reaction. Flow cytometry is capable of sensitive and quantitative fluorescence measurements of individual particles without the need to separate free from particle-bound label. Analysis rates are very high (hundreds to thousands of particles per second), and multiple fluorescence and light scatter signals can be detected simultaneously.

In preferred embodiments of the above aspects of the invention, the added nucleotide is a labeled ddNTP. In particular embodiments, the label can be a fluorescent label (e.g., 6-FAM, Cy5®, Cy3®, FITC, rhodamine, lanthamide phosphors, Texas red). Fluorescent dyes are detected through exposure of the label to a photon of energy of one wavelength, supplied by an external source such as an incandescent lamp or laser, causing the fluorophore to be transformed into an excited state. The fluorophore then emits the absorbed energy in a longer wavelength than the excitation wavelength which can be measured as fluorescence by standard instruments containing fluorescence detectors. Exemplary fluorescence instruments include spectrofluorometers and microplate readers, fluorescence microscopes, fluorescence scanners, and flow cytometers.

In addition to labeling nucleic acids with fluorescent dyes, the invention can be practiced using any apparatus or methods to detect detectable labels associated with nucleic acids of a sample, an individual member of the nucleic acids of a sample. Devices and methods for the detection of multiple fluorophores are well known in the art, see, e.g., U.S. Pat. Nos. 5,539,517; 6,049,380; 6,054,279; 6,055,325; and 6,294,331. Any known device or method, or variation thereof, can be used or adapted to practice the methods of the invention, including array reading or “scanning” devices, such as scanning and analyzing multicolor fluorescence images; see, e.g., U.S. Pat. Nos. 6,294,331; 6,261,776; 6,252,664; 6,191,425; 6,143,495; 6,140,044; 6,066,459; 5,943,129; 5,922,617; 5,880,473; 5,846,708; 5,790,727; and, the patents cited in the discussion of arrays, herein. See also published U.S. Patent Application Nos. 20010018514; 20010007747; and published international patent applications Nos. WO0146467 A; WO9960163 A; WO0009650 A; WO0026412 A; WO0042222 A; WO0047600 A; and WO0101144 A.

Charge-coupled devices, or CCDs, are used in microarray scanning systems can be used in the practice of the methods described herein. Color discrimination can also be based on 3-color CCD video images; these can be performed by measuring hue values. Hue values are introduced to specify colors numerically. Calculation is based on intensities of red, green and blue light (RGB) as recorded by the separate channels of the camera. The formulation used for transforming the RGB values into hue, however, simplifies the data and does not make reference to the true physical properties of light. Alternatively, spectral imaging can be used; it analyzes light as the intensity per wavelength, which is the only quantity by which to describe the color of light correctly. In addition, spectral imaging can provide spatial data, because it contains spectral information for every pixel in the image. Alternatively, a spectral image can be made using brightfield microscopy, see, e.g., U.S. Pat. No. 6,294,331.

Probe Hybridization

Amplified nucleic acids can be detected by hybridization with a specific probe. Probes, complementary to a portion of the amplified target sequence can be used to detect amplified fragments. Hybridization can be detected in real time or in non-real time. Amplified nucleic acids for each of the target sequences can be detected simultaneously (i.e., in the same reaction vessel) or individually (i.e., in separate reaction vessels). In some embodiments, the amplified DNA is detected simultaneously, using two or more distinguishably-labeled, gene-specific probes, one which hybridizes to the first target sequence, one which hybridizes to the second target sequence, and so forth.

The probe can be detectably labeled by methods known in the art. Useful labels include, e.g., fluorescent dyes (e.g., Cy5, Cy3, FITC, rhodamine, lanthamide phosphors, Texas red, FAM, JOE, Cal Fluor Red 610®, Quasar 670®), ³²P, ³⁵S, ³H, ¹⁴C, ¹²⁵I, ¹³¹I, electron-dense reagents (e.g., gold), enzymes, e.g. as commonly used in ELISA (e.g., horseradish peroxidase, beta-galactosidase, luciferase, alkaline phosphatase), calorimetric labels (e.g., colloidal gold), magnetic labels (e.g., Dynabeads™), biotin, dioxigenin, or haptens and proteins for which antisera or monoclonal antibodies are available. Other labels include ligands or oligonucleotides capable of forming a complex with the corresponding receptor or oligonucleotide complement, respectively. The label can be directly incorporated into the nucleic acid to be detected, or it can be attached to a probe (e.g., an oligonucleotide) or antibody that hybridizes or binds to the nucleic acid to be detected.

Suitable fluorescent moieties include the following fluorophores known in the art: 4-acetamido-4′-isothiocyanatostilbene-2,2′disulfonic acid, acridine and derivatives (acridine, acridine isothiocyanate) Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes), 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Black Hole Quencher™ (BHQ™) dyes (biosearch Technologies), BODIPY® R-6G, BOPIPY® 530/550, BODIPY® FL, Brilliant Yellow, coumarin and derivatives (coumarin, 7-amino-4-methylcoumarin (AMC, Coumarin 120), 7-amino-4-trifluoromethylcouluarin (Coumarin 151)), Cy2®, Cy3®, Cy3.5®, Cy5®, Cy5.5®, cyanosine, 4′,6-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin, diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-(4′-dimethylaminophenylazo)benzoic acid (DABCYL), 4-dimethylaminophenylazophenyl-4′-isothiocyanate (DABITC), Eclipse™ (Epoch Biosciences Inc.), eosin and derivatives (eosin, eosin isothiocyanate), erythrosin and derivatives (erythrosin B, erythrosin isothiocyanate), ethidium, fluorescein and derivatives (5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET)), fluorescamine, IR144, IR1446, Malachite Green isothiocyanate, 4-methylumbelliferone, ortho cresolphthalein, nitrotyrosine, pararosaniline, Phenol Red, B-phycoerythrin, R-phycoerythrin, o-phthaldialdehyde, Oregon Green®, propidium iodide, pyrene and derivatives (pyrene, pyrene butyrate, succinimidyl 1-pyrene butyrate), QSY® 7, QSY® 9, QSY® 21, QSY® 35 (Molecular Probes), Reactive Red 4 (Cibacron® Brilliant Red 3B-A), rhodamine and derivatives (6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine B, sulforhodamine 101, sulfonyl chloride derivative of sulforhodamine 101 (Texas Red)), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives.

Other fluorescent nucleotide analogs can be used, see, e.g., Jameson, 278 Meth. Enzymol. 363-390 (1997); Zhu, 22 Nucl. Acids Res. 3418-3422 (1994). U.S. Pat. Nos. 5,652,099 and 6,268,132 also describe nucleoside analogs for incorporation into nucleic acids, e.g., DNA and/or RNA, or oligonucleotides, via either enzymatic or chemical synthesis to produce fluorescent oligonucleotides. U.S. Pat. No. 5,135,717 describes phthalocyanine and tetrabenztriazaporphyrin reagents for use as fluorescent labels.

A useful detectable moiety is a cyanine dye such as Cy-5 and Cy-3, FAM, HEX, and the like. A detectable moiety can include more than one chemical entity such as in fluorescent resonance energy transfer (FRET). Resonance transfer results in overall enhancement of the emission intensity. For instance, see Ju et. al. (1995) Proc. Nat'l Acad. Sci. (USA) 92: 4347. To achieve resonance energy transfer, the first fluorescent molecule (the “donor” fluorophore) absorbs light and transfers it through the resonance of excited electrons to the second fluorescent molecule (the “acceptor” fluorophore). In one approach, both the donor and acceptor dyes can be linked together and attached to the primer. Methods to link donor and acceptor dyes to a nucleic acid have been described previously, for example, in U.S. Pat. No. 5,945,526 to Lee et al. Donor/acceptor pairs of dyes that can be used include, for example, fluorescein/tetramethylrohdamine, IAEDANS/fluoroescein, EDANS/DABCYL, fluorescein/fluorescein, BODIPY FL/BODIPY FL, and Fluorescein/QSY 7 dye. See, e.g., U.S. Pat. No. 5,945,526 to Lee et al. Many of these dyes also are commercially available, for instance, from Molecular Probes Inc. (Eugene, Oreg.). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

The detectable label can be incorporated into, associated with or conjugated to a nucleic acid. The label can be attached by spacer arms of various lengths to reduce potential steric hindrance or impact on other useful or desired properties. See, e.g., Mansfield, 9 Mol. Cell. Probes 145-156 (1995). Detectable labels can be incorporated into nucleic acids by covalent or non-covalent means, e.g., by transcription, such as by random-primer labeling using Klenow polymerase, or nick translation, or amplification, or equivalent as is known in the art. For example, a nucleotide base is conjugated to a detectable moiety, such as a fluorescent dye, and then incorporated into nucleic acids during nucleic acid synthesis or amplification.

Signal amplification can be achieved using labeled dendrimers as the detectable moiety (see, e.g., Physiol Genomics 3:93-99, 2000). Fluorescently labeled dendrimers are available from Genisphere (Montvale, N.J.). These can be chemically conjugated to the primers by methods known in the art.

The detection of the target nucleic acids can be accomplished by means of so called Invader™ technology (available from Third Wave Technologies Inc. Madison, Wis.). In this assay, a specific upstream “invader” oligonucleotide and a partially overlapping downstream probe together form a specific structure when bound to complementary DNA template. This structure is recognized and cut at a specific site by the Cleavase enzyme, and this results in the release of the 5′ flap of the probe oligonucleotide. This fragment then serves as the “invader” oligonucleotide with respect to synthetic secondary targets and secondary fluorescently labeled signal probes contained in the reaction mixture. This results in specific cleavage of the secondary signal probes by the Cleavase enzyme. Fluorescence signal is generated when this secondary probe, labeled with dye molecules capable of FRET, is cleaved Cleavases have stringent requirements relative to the structure formed by the overlapping DNA sequences or flaps and can, therefore, be used to specifically detect single base pair mismatches immediately upstream of the cleavage site on the downstream DNA strand. See Ryan D et al. Molecular Diagnosis 4(2): 135-144 (1999) and Lyamichev V et al. Nature Biotechnology 17:292-296 (1999), see also U.S. Pat. Nos. 5,846,717 and 6,001,567.

Melting Curve Analysis

Melting curve analysis can be used to detect an amplification product. One advantage is the possible use of fewer dyes if desired. Melting curve analysis involves determining the melting temperature of a nucleic acid amplicon by exposing the amplicon to a temperature gradient and observing a detectable signal from a fluorophore. Melting curve analysis is based on the fact that a nucleic acid sequence melts at a characteristic temperature called the melting temperature (Tₘ), which is defined as the temperature at which half of the DNA duplexes have separated into single strands. The melting temperature of a DNA depends primarily upon its nucleotide composition. Thus, DNA molecules rich in G and C nucleotides have a higher Tₘ than those having an abundance of A and T nucleotides.

Where a fluorescent dye is used to determine the melting temperature of a nucleic acid in the method, the fluorescent dye emits a signal that can be distinguished from a signal emitted by any other of the different fluorescent dyes that are used to label the oligonucleotides. In some embodiments, the fluorescent dye for determining the melting temperature of a nucleic acid can be excited by different wavelength energy than any other of the different fluorescent dyes that are used to label the oligonucleotides. In some embodiments, the second fluorescent dye for determining the melting temperature of the detected nucleic acid is an intercalating agent. Suitable intercalating agents include, but are not limited to SYBR™ Green I dye, SYBR™ dyes, Pico Green, SYTO dyes, SYTOX dyes, ethidium bromide, ethidium homodimer-1, ethidium homodimer-2, ethidium derivatives, acridine, acridine orange, acridine derivatives, ethidium-acridine heterodimer, ethidium monoazide, propidium iodide, cyanine monomers, 7-aminoactinomycin D, YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, and mixture thereof.

By detecting the temperature at which the fluorescence signal is lost, the melting temperature can be determined. For example, amplified target nucleic acids may have a melting temperature that differs by at least about 1° C., more preferably by at least about 2° C., or even more preferably by at least about 4° C. from the melting temperature of any other amplified target nucleic acids. By observing differences in the melting temperature(s) of the gene or gene fragment targets from the respective amplification products, the presence or absence of the target sequence in the sample can be confirmed.

Preparation of an Internal Control

As a quality control measure, an internal amplification control (IC) can be included in one or more samples to be extracted and amplified. The skilled artisan will understand that any detectable sequence that is not derived from the target sequences can be used as the control sequence. These controls can be mixed with the sample (or with purified nucleic acids isolated from the sample), and amplified with sample nucleic acids using a pair complementary to the control sequence. If PCR amplification is successful, the internal amplification control amplicons can then be detected and differentiated from the target amplicons. Additionally, if included in the sample prior to purification of nucleic acids, the control sequences can also act as a positive purification control.

All publications, patent applications, issued patents, and other documents referred to in the present disclosure are herein incorporated by reference as if each individual publication, patent application, issued patent, or other document were specifically and individually indicated to be incorporated by reference in its entirety. Definitions that are contained in text incorporated by reference are excluded to the extent that they contradict definitions in this disclosure.

### EXAMPLES

The methods are further illustrated by the following examples, which should not be construed as limiting in any way.

### Example 1

### Materials and Methods

Exemplary reagents that can be used in the following Examples are listed in Table 4.

TABLE 4
Reagents

dNTP set, ultrapure, 100 mM solution (Pharmacia, #27-2035-01)
Water, molecular biology grade (BioWhittaker, #16-001Y)
HotStarTaq ™ PCR Core Kit (Qiagen 203203 or 203205)
(HotStarTaq ^{™} enzyme, 25 mMg⁺⁺, M10X buffer & 5X Q Solution)
FastStartTaq ™ PCR Kit (Roche, 2 032 953) (FastStartTaq ™ enzyme,
25 m, Mg⁺⁺, 10X PCR Reaction Buffer & 5X GCRICH Solution)
PCR primers, 0.2 or 1.0 micromole scale synthesis, desalted
(synthesized by Operon)
Alkaline Phosphatase, Calf Intestinal (Promega, #M1821)
Exonuclease I (USB Corporation, #70073X)
ABI SNaPshot Multiplex kit (Applied Biosystems, #4323161)
Primer extension primers, 0.2 or 1.0 micromole scale synthesis,
HPLC purification (synthesized by Operon)
Rox 1000 Size Standard (Abbott Molecular, #6L44-08)
ABI GeneScan-120 LIZ Size Standard (Applied Biosystems, #4322362)
ABI Running Buffer (10X) with EDTA (Applied Biosystems, #402824)
ABI Hi-Di Formamide (Applied Biosystems, #4311320)
3100 POP-4 Polymer (Applied Biosystems, #4316355)

The 25 mM dNTPs were prepared as follows: 50 μL of each of a 100 mM stock solution of dATP, dCTP, dGTP, and dTTP were added to a sterile microcentrifuge tube and vortexed for 2 seconds to mix, and spun in microcentrifuge at maximum speed for 2 seconds. Samples were not refrozen more than five times.

Preparation of DNA

Whole blood (5 cc) was collected and stored in either an EDTA or ACD tube. Genomic DNA was extracted for use in SNaPshot PCR Amplification and in Deletion Duplication PCR Amplification. Each sample was extracted using the Qiagen 9604 Robot. Each sample was lysed in the presence of Qiagen Protease and Buffer AL under highly denaturing conditions. The lysate buffering conditions were adjusted to allow binding of the DNA to the QIAmp membrane by the addition of ethanol. DNA was absorbed on the silica-gel membrane using vacuum. Salt and pH conditions in the lysate ensured that impurities which could inhibit PCR were not retained on the membrane. DNA bound to the membrane was washed using a vacuum and centrifugation. The DNA was eluted in 200 μL of buffer. The eluted DNA was ready for use in PCR. Degraded DNA samples, or DNA samples that were contaminated by inhibitors of PCR were not used.

### Example 2

### SNaPshot PCR Amplification

SNaPshot PCR amplification was used to amplify fragments of interest for 10 missense mutations. The primers used, size of the amplified fragments, and the corresponding allele(s) detected for each fragment, are shown in Table 5.

TABLE 5
SNaPshot Primers and Fragments

| Primer | | | |
|---|---|---|---|
| SEQ ID | Fragment Nucleotide | Fragment Size | Mutation(s) |
| NOs | Sequence (FIG. 1) | (targeted exon) | Detected |
| 29, 30 | 1450-2226 | 777 bp (exon 1) | *9 |
| 27, 28 | 3521-4266 | 746 bp (exons 3 and 4) | *2, *6, *20 |
| 25, 26 | 4267-5521 | 1255 bp (exon 5) | *11 |
| 23, 24 | 6749-7621 | 873 bp (exons 7 and | *17, *18 |
| | | part of 8) | |
| 21, 22 | 7972-8875 | 904 bp (exon 9) | *5, *7, *8 |

The SNaPshot primer pair mix, PCR master mix, and PCR reagents are shown in Table 6, Table 7, and Table 8 respectively.

TABLE 6
SNaPshot Primer Pair Mix

| | Cocktail ×1 | Cocktail ×1,000 | Final | |
|---|---|---|---|---|
| Primers (100 μM) | (μL) | (μL) | Concentration | |
| 2A6UTR-AS1 | 0.075 | 75 | 0.3 | μM |
| 2A6E9R3 | 0.075 | 75 | 0.3 | μM |
| 2A6E78F3 | 0.0375 | 37.5 | 0.15 | μM |
| 2A6E78R2 | 0.0375 | 37.5 | 0.15 | μM |
| 2A6E5F2 | 0.2 | 200 | 0.8 | μM |
| 2A6E5R2 | 0.2 | 200 | 0.8 | μM |
| 2A6E34F2 | 0.01875 | 18.75 | 0.075 | μM |
| 2A6E34R2 | 0.01875 | 18.75 | 0.075 | μM |
| 2A6ElF2 | 0.025 | 25 | 0.1 | μM |
| 2A6E1R2 | 0.025 | 25 | 0.1 | μM |
| Total | 0.7125 | 712.5 | | |

TABLE 7
SNaPshot PCR Master Mix

| | Cocktail ×1 | Cocktail ×1,000 | Final |
|---|---|---|---|
| PCR Mix | (μL) | (μL) | Concentration |
| Primer Pair Mix | .7125 | 712.5 | Varies |
| 10X Quiagen | 2.5 | 2,500 | 1X |
| PCR Buffer | | | |
| 25 mM MgCl | .5 | 500 | .5 mM |
| 25 mM dNTP mix | .4 | 400 | .4 mM |
| Sterile dH₂O | 18.6875 | 18,687.5 | — |
| Total | 22.8 | 22,800 | |

TABLE 8
SNaPshot PCR Reagents

| Reagents | ×1 (μL) | ×10 (μL) | ×20 (μL) |
|---|---|---|---|
| Master Mix | 22.8 | 228 | 456 |
| HotStarTaq | 0.2 | 2 | 4 |
| Total | 23 | 230 | 460 |

If a DNA sample was extracted with the phenol/chloroform method, it was diluted in sterile water to a concentration of 20-40 ng/(μL).

The PCR reagent mixture shown in Table 8 was added to a PCR plate followed by the addition of 2 μL patient DNA and 2 μL control DNA to each appropriate well. The wells were sealed and spun for 15 seconds at 1600-3000 rpm in a Sorval T6000D centrifuge or equivalent. The CYP2A6 thermal cycler program parameters are listed in Table 9. The PCR plate was placed in the thermal cycler once the block temperature reached 60° C.-80° C.

TABLE 9
PCR Cycling Parameters (ABI 9700 or MJ PTC 200 or 225)

| Step | Temperature | Time | |
|---|---|---|---|
| 1 | 95° C. | 15 | min |
| 2 | 95° C. | 30 | sec |
| 3 | 58° C. | 1 | min |
| 4 | 72° C. | 3 | min |
| 5 | Go to Step 2 | 35 more times | |
| 6 | 72° C. | 5 | min |
| 7 | 4° C. | Hold | |
| 8 | End | | |

No internal control fragments were added to the SNaPshot assay. If a peak failed, the assay was repeated.

SNaPshot CIP and Exonuclease I Digestion

The multiplex PCR fragments were treated with CIP and Exol to remove unincorporated dNTPs and PCR primers. The amount of CIP+Exol digestion cocktail, prepared fresh each time prior to use, was based on the number of samples to be digested as is shown in Table 10.

TABLE 10
SNaPshot First CIP + ExoI Digestion Cocktail

| REAGENTS | ×1 (μL) | ×10 (μL) | ×20 (μL) |
|---|---|---|---|
| CIP (1 unit/μl) | 2 | 20 | 40 |
| Exol (10 unit/μl) | 0.2 | 2 | 4 |
| Sterile dH₂0 | 12.8 | 128 | 256 |
| Total | 15 | 150 | 300 |

Into each well of a new 96-well plate, 15 μL of the CIP+Exol digestion cocktail was aliquoted to which was added 5 μL of the SNaPshot PCR product. The plate was sealed, vortexed, centrifuged briefly, and then placed in the thermal cycler. The program parameters are shown in Table 11.

TABLE 11
SNaPshot Thermal Cycler Program Parameters after CIP +
ExoI Digestion (ABI 9700 or MJ PTC 200 or 225)

| Step | Temperature | Time | |
|---|---|---|---|
| 1 | 37° C. | 2 | hr. |
| 2 | 75° C. | 15 | min. |

| | | |
|---|---|---|
| 3 | 4° C. | Hold |
| 4 | End | |

SNaPshot Primer Extension

The SNaPshot primer extension primer mix and SNaPshot primer extension master mix are shown in Table 12 and Table 13 respectively.

TABLE 12
SNaPshot Primer Extension Primer Mix

| | | | Final |
|---|---|---|---|
| Extension Primer | ×1 (μL) | ×1,000 (μL) | Concentration |
| 2A6*6R2 | 0.015 | 15 | 0.15 μM |
| 2A6*5R2 | 0.02 | 20 | 0.2 μM |
| 2A6* 18R2 | 0.02 | 20 | 0.2 μM |
| 2A6*2F2 | 0.03 | 30 | 0.3 μM |
| 2A6* 17R2 | 0.02 | 20 | 0.2 μM |
| 2A6* 11F3 | 0.02 | 20 | 0.2 μM |
| 2A6*20R2 | 0.02 | 20 | 0.2 μM |
| 2A6*9F3 | 0.02 | 20 | 0.2 μM |
| 2A6*8F2 | 0.02 | 20 | 0.2 μM |
| 2A6*7R3 | 0.02 | 20 | 0.2 μM |
| Sterile dH₂0 | 1.795 | 1,795 | |
| Total | 2 | 2,000 | |

TABLE 13
SNaPshot Primer Extension Master Mix

| Reagents | ×1 (μL) | ×10 (μL) | ×20 (μL) |
|---|---|---|---|
| SNaPshot Ready Mix¹ | 2.5 | 25 | 50 |
| 5X Sequencing Buffer | 2.5 | 25 | 50 |
| Primer Extension Primer Mix | 2 | 20 | 40 |
| Total | 7 | 70 | 140 |

¹The SNaPshot Ready mix was comprised of AmpliTaq DNA polymerase FS, fluorescently labeled ddNTPs and reaction buffer. The tags on the ddNTPs were as follows: A was labeled with dR6g, C was labeled with dTAMRA, G was labeled with dR110, and T was labeled with dROX.

Into each well of a new 96-well plate, 7 μL of the SNaPshot primer extension master mix was aliquoted to which was added 3 μL of the first CIP+Exol digested PCR products. The plate was sealed, vortexed, centrifuged briefly, and then placed in the thermal cycler. The program parameters are shown in Table 14.

TABLE 14
SNaPshot Primer Extension Thermal Cycler Program
Parameters (ABI 9700 or MJ PTC 200 or 225)

| Step | Temperature | Time | |
|---|---|---|---|
| 1 | 96° C. | 10 | sec. |
| 2 | 50° C. | 5 | sec |
| 3 | 60° C. | 30 | sec |
| 4 | Go To Step 1 | 24 | Cycles |
| 5 | 4° C. | Hold | |
| 6 | End | | |

SNaPshot Second CIP Digestion

The primer extension products were spun down for 15 seconds at approximately 1,500-3,000 rpm in a Sorvall T6000D centrifuge or equivalent. A second CIP digestion cocktail was prepared for the appropriate number of samples to be digested as is shown in Table 15.

TABLE 15
SNaPshot Second CIP Digestion Cocktail

| Reagent | ×1 (μL) | ×10 (μL) | ×20 (μL) |
|---|---|---|---|
| CIP (1 unit/μl) | 1 | 10 | 20 |
| Water | 1 | 10 | 20 |
| Total | 2 | 20 | 40 |

To each primer extension product, 2 mL of the second CIP digestion cocktail was added. The plate was sealed, vortexed, centrifuged briefly, and then placed in the thermal cycler. The program parameters are shown in Table 16.

TABLE 16
SNaPshot Thermal Cycler Program Parameters after Second
CIP Digestion (ABI 9700 or MJ PTC 200 or 225)

| Step | Temperature | Time | |
|---|---|---|---|
| 1 | 37° C. | 1 | hour |
| 2 | 75° C. | 15 | min. |

| | | |
|---|---|---|
| 3 | 4° C. | Hold |
| 4 | End | |

SNaPshot Capillary Electrophoresis and Detection on Agarose Gels

The fragments were detected on an automated DNA sequencer (e.g., ABI PRISM 3100 Genetic Analyzer—Applied Biosystems). The ABI 3100 10× buffer was diluted 1:10 in H₂O (e.g., 5 mL of buffer+45 mL H₂O). The second CIP digestion products were spun down for approximately 15 seconds at approximately 1,500-3,000 rpm in a Sorvall T6000D centrifuge or equivalent. The appropriate amount of Loading Cocktail for the number of samples to be digested was prepared according to Table 17.

TABLE 17
SNaPshot Loading Cocktail

| Reagents | ×1 (μL) | ×10 (μL) | ×20 (μL) |
|---|---|---|---|
| Hi-Di Formamide | 10.5 | 105 | 210 |
| GS 120 Liz | 0.5 | 5 | 10 |
| Total | 11 | 110 | 220 |

The second CIP-digested primer extension product was diluted 1:10 by adding 108 μL water to 12 μL of the second digest. Then 2 μL of the diluted product was mixed with 11 μL of the Loading Mix in a new ABI Optical plate. The plate was vortexed, centrifuged, heated at 93° C.-97° C. for 5 minutes, and then immediately placed on ice for 5 minutes or until use. The plate was loaded onto the ABI 3100 Genetic Analyzer to detect the fluorescently labeled oligonucleotides both by size and by fluorescent label. GenoTyper 3.7 software or higher was used to perform SNaPshot data analysis. The expected allele peak size and color are shown in Table 18.

TABLE 18
Expected Allele Peak Size and Color

| | | Wild type allele | | | Mutant allele | | |
|---|---|---|---|---|---|---|---|
| | Primer | Extended | | | Extended | | |
| Mutation | size (bp) | nucleotide | Color | Size (bp)* | nucleotide | Color | Size (bp)* |
| *5 | 20 | G | Blue | 23.0-24.0 | T | Red | 25.65-26.75 |
| *2 | 20 | A | Green | 27.0-28.0 | T | Red | 27.0-28.0 |
| *6 | 30 | G | Blue | 31.0-32.0 | A | Green | 31.75-32.75** |
| *18 | 32 | A | Green | 35.0-36.0 | T | Red | 35.5-36.5** |
| *7 | 36 | T | Red | 39.0-40.0 | C | Black | 37.6-38.6 |
| *8 | 38 | C | Black | 43.0-44.0 | A | Green | 43.5-44.5 |
| *9 | 41 | A | Green | 47.0-48.0 | C | Black | 46.7-47.4 |
| *11 | 47 | A | Green | 51.5-52.5 | G | Blue | 49-51.5** |
| *17 | 53 | G | Blue | 54.5-55.5 | A | Green | 55.7-56.5 |
| *20 | 55 | A | Green | 57.5-59.0 | G | Blue | 57.4-58.4 |

*Size of the primer extension product is affected by mobility shift caused by the incorporated fluorescent dye labeled ddNTP.
**Size of these mutants is not exactly known due to the fact that these mutations have not been seen yet.

### Example 3

### Deletion Duplication PCR Amplification

Multiplex PCR amplification was used to amplify fragments of interest to detect gene deletion, duplication, and/or rearrangement. The primers used, size of the amplified fragments, and the corresponding allele(s) detected for each fragment are shown in Table 19.

TABLE 19
Gene Deletion Duplication Primers and Fragments

| Primer | | | |
|---|---|---|---|
| SEQ ID | Fragment Nucleotide | Fragment Size | Mutation(s) |
| NOs | Sequence (FIG. 1) | (targeted exon) | Detected |
| 29, 40 | 1450-2226 | 777 bp (exon 1) | *4, *12 |
| 28, 39 | 3521-4266 | 746 bp (exons 3 and 4) | *4 |
| 24, 38 | 6749-7621 | 873 bp (exons 7 and | *4 |
| | | part of 8) | |
| 22, 37 | 7972-8875 | 904 bp (exon 9) | *4 |

The deletion duplication primer pair mix, PCR master mix, and PCR reagents are shown in Table 20, Table 21, and Table 22 respectively.

TABLE 20
Deletion Duplication Primer Pair Mix

| Primers | Cocktail ×1 | Cocktail ×1,000 | Final | |
|---|---|---|---|---|
| (100 μM) | (μL) | (μL) | Concentration | |
| 2A6E1F2 | 0.08 | 80 | 0.32 | μM |
| 2A6E1R2-FAM | 0.08 | 80 | 0.32 | μM |
| 2A6E34F2-FAM | 0.02 | 20 | 0.08 | μM |
| 2A6E34R2 | 0.02 | 20 | 0.08 | μM |
| 2A6E78F3-FAM | 0.25 | 250 | 1.0 | μM |
| 2A6E78R2 | 0.25 | 250 | 1.0 | μM |
| 2A6UTR-AS1-FAM | 0.6 | 600 | 2.4 | μM |
| 2A6E9R3 | 0.6 | 600 | 2.4 | μM |
| 2A7Fben-FAM | 0.02 | 20 | 0.08 | μM |
| 2A7Rben | 0.02 | 20 | 0.08 | μM |
| 1B1SNPEx3R5′-FAM | 0.01 | 10 | 0.04 | μM |
| 1B1SNPEx3R 5′ | 0.01 | 10 | 0.04 | μM |
| CFDEL22F-FAM | 0.5 | 500 | 2.0 | μM |
| CFDEL23R | 0.5 | 500 | 2.0 | μM |
| Total | 2.96 | 2,960 | | |

TABLE 21
Deletion Duplication PCR Master Mix

| | Cocktail ×1 | Cocktail ×1,000 | Final |
|---|---|---|---|
| PCR Mix | (μL) | (μL) | Concentration |
| Primer Pair Mix | 2.96 | 2,960 | Varies |
| 10X Roche PCR | 2.5 | 2,500 | 1X |
| Buffer | | | |
| (without MgCl) | | | |
| 25 mM MgCl | 4 | 4,000 | 4 mM |
| 25 mM dNTP mix | 0.4 | 400 | 0.4 mM |
| Sterile dH₂0 | 12.94 | 12,940 | — |
| Total | 22.8 | 22,800 | |

TABLE 22
Deletion Duplication PCR Reagents

| Reagents | ×1 (μL) | ×10 (μL) | ×20 (μL) |
|---|---|---|---|
| Master Mix | 22.8 | 228 | 456 |
| FastStartTaq | 0.2 | 2 | 4 |
| Total | 23 | 230 | 460 |

The PCR reagent mixture shown in Table 22 was added to a PCR plate followed by the addition of 2 μL patient DNA and 2 μL control DNA to each appropriate well.

The wells were sealed and spun for 15 seconds at 1600-3000 rpm in a Sorval T6000D centrifuge or equivalent. The CYP2A6 thermal cycler program parameters are listed in Table 23. The PCR plate was placed in the thermal cycler once the block temperature reached 60° C.-80° C.

TABLE 23
PCR Cycling Parameters (ABI 9700 or MJ PTC 200 or 225)

| Step | Temperature | Time | |
|---|---|---|---|
| 1 | 95° C. | 15 | min |
| 2 | 94° C. | 30 | sec |
| 3 | 58° C. | 1 | min |
| 4 | 72° C. | 3 | min |
| 5 | Go to Step 2 | 23 more times | |
| 6 | 72° C. | 5 | min |
| 7 | 4° C. | Hold | |
| 8 | End | | |

Along with the CYP2A6 PCR fragments, control fragments from the genes of CYP2A7, CYP1B1 (exon 3) and CFTR (exon 22) were added to the multiplex. Either the forward or reverse primer for each pair had 6-FAM attached to it.

Deletion Duplication Capillary Electrophoresis and Detection on Agarose Gels

A multiplex semi-quantitative fluorescent PCR (SQF PCR) was used for the detection of deletions and duplications. The cycling conditions for the PCR were stopped when the reaction was still in the linear phase. The fragments were visualized on an automated DNA sequencer (e.g., ABI PRISM 3100 Genetic Analyzer). The ABI 3100 10× buffer was diluted 1:10 in H₂O (e.g., 5 mL of buffer+45 mL H₂O). The PCR products were spun down for approximately 15 seconds at approximately 1,500-3,000 rpm in a Sorvall T6000D centrifuge or equivalent. The appropriate amount of Loading Cocktail for the number of samples to be digested was prepared according to Table 24.

TABLE 24
Deletion Duplication Loading Cocktail

| Reagents | ×1 (μL) | ×10 (μL) | ×20 (μL) |
|---|---|---|---|
| Hi-Di Formamide | 10 | 100 | 200 |
| Rox 1000 | 1 | 10 | 20 |
| Total | 11 | 110 | 220 |

In a new ABI Optical plate, 2 μL of the PCR product was mixed with 11 μL of the Loading Cocktail. The plate was vortexed, centrifuged, heated at 93°-97° C. for 3 minutes, and then immediately placed on ice for 3 minutes or until use. The plate was loaded onto the ABI 3100 Genetic Analyzer to detect the fluorescently labeled oligonucleotides. GeneMapper software 3.0 or higher was used to perform deletion duplication data analysis. The peak height, in relative fluorescent intensity, for each fragment from the CYP2A6 gene were compared to the three internal controls. If there was roughly a 50% reduction in peak height for the CYP2A6 fragment, as compared to the controls, then a deletion was present. If there was roughly a 30-50% increase in peak height for the CYP2A6 fragment, as compared to the controls, then a duplication was present.

### Example 4

### Interpretation of Data and Quality Controls

A person tested could be negative for all mutations examined. However, this result did not rule out the possibility of another mutation in the CYP2A6 gene that was not assayed for. In the deletion duplication assay, a patient could be found to be positive for one or two copies of the *4 deletion or the *12 rearrangement mutations. The *4 deletion was shown as a decrease in the signals for exons 1, 3, 4, 7, 8 and 9 but not in the case of *4D which showed exon 9 at normal levels. The *12 rearrangement mutation was shown as a decrease in exon 1 only. The *12/wt mutation appeared as a 50% reduction in the exon 1 fragment only. All other fragments were the same height as a wt/wt sample. A *12/*12 sample appeared as no signal at all for exon 1 while all other fragments were the same height as a wt/wt sample. The *4D/wt appeared as a 50% reduction in exons 1, 3-4, and 7-8. The exon 9 fragment remained the same height as a wt/wt sample. The *4D/*4D appeared with no peaks for exons 1, 3-4 and 7-8 fragments with the exon 9 fragment at the same height as a wt/wild-type sample. The *4A/wt and *4B/wt both appeared as a 50% reduction for all CYP2A6 fragments (exons 1, 3-4, 7-8, and 9). The *4A/*4A and *4B/*4B only showed peaks for the internal controls. Patients who were positive with two copies of the same mutation or had one copy each of two different mutations were considered poor metabolizers.

Repeat Criteria: if a patient sample yielded no interpretable results, the assay was repeated. Samples on batches that failed the Quality Control criteria were reported and on occasion re-assayed.

Quality Control—Controls Used

Positive Controls: wt/wt and rotating positive controls that were available, or equivalent control materials were included in every run. They were used to show the expected genotype.

Negative Controls

No Sample (NS) Control: The NS control was a reagent blank that was included on every extraction plate. It included all reagents and processing used to prepare sample DNA but without any starting DNA. The control was used to test for contamination of the reagents used to extract the patient's DNA. An NS control was included on each assay run. If patient samples from different extractions were included on a run, an NS control for each extraction was included.

No DNA (ND) Control: The ND control was used to test for contamination of the PCR reagents and enzymes. The ND control (consisting of a PCR kit and polymerase mix used for the assay run—dH₂O instead of DNA) was placed at the end of each run.

Negative controls were used to display no significant amplification and/or fluorescent signal. If the reagent blank (NS control) showed evidence of significant amplification, all the patient samples associated with that NS control were potentially contaminated. If the minus DNA control (D control) yielded significant amplification, the PCR amplification reagents were potentially contaminated. Possible sources of contamination included the PCR Master Mix and/or the PCR stock reagents used to prepare the Master Mix. In such a scenario, the specimens might need to be re-extracted and re-assayed (NS) and the entire assay repeated (D).

Negative controls were used to display no significant fluorescent signal upon electrophoresis on an ABI 3100 genetic analyzer. If the NS control showed evidence of significant fluorescence, all the patient samples associated with that NS control were potentially contaminated.

Positional Control (QC Blank): One or more QC blanks (e.g., no DNA) were placed randomly within each extraction plate to ensure that the results reflected the correct positioning and orientation of the extraction/PCR plate for the assay.

The appropriate control DNAs were included on each PCR run. The controls were positioned at the end of the PCR plate, following the patient samples. The controls were added to the PCR plate, only after all of the patient samples had been added. The wt/wt control DNA were from the same extraction methodology as the patient samples tested. If the patient samples were extracted on multiple extraction methodologies, multiple wt/wt controls were also required.

Unless otherwise defined, all technical and scientific terms used herein have the same meaning as commonly understood by one of ordinary skill in the art.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the invention claimed.

Thus, it should be understood that although the present invention has been specifically disclosed by preferred embodiments and optional features, modification, improvement, and variation of the inventions disclosed can be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. Applicants reserve the right to physically incorporate into this application any and all materials and information from any such articles, patents, patent applications, or other physical and electronic documents. In case of conflict, the present specification, including definitions, will control.

Other embodiments are set forth within the following claims.

What is claimed is:

1. A method for determining the presence or absence of one or more mutations in the CYP2A6 gene from nucleic acids in a sample said method comprising:
(a) using at least one oligonucleotide capable of hybridizing to one or more of the sequences of SEQ ID NOs: 1-10 or complements thereof,
(b) amplifying with said oligonucleotide at least one fragment of the CYP2A6 gene, containing the site of the mutation, and
(c) detecting the presence or absence of said one or more mutations in the CYP2A6 gene,
wherein said oligonucleotide is capable of specifically amplifying the CYP2A6 gene but not the CYP2A7 gene or pseudogenes of CYP2A6 and CYP2A7.

2. The method of claim 1, wherein said oligonucleotide comprises at least one of the sequences of SEQ ID NOs: 11-20 or complements thereof, with a proviso that the 5′ end of the sequences of SEQ ID NOs: 11-20 and complements thereof comprises 1 to 6 optional nucleotides as shown in Table 2.

3. The method of claim 1 wherein said oligonucleotide comprises a sequence selected from the group consisting of the sequences of SEQ ID NOs: 21-30 and complements thereof.

4. The method of claim 1, wherein said amplifying is accomplished with a polymerase chain reaction (PCR).

5. The method of claim 1 further comprising performing single nucleotide primer extension to detect the identity of the nucleotide added, wherein the identity of the nucleotide indicates the presence or absence of said mutation in the CYP2A6 gene.

6. The method of claim 5, further comprising detecting the presence or absence of said one or more mutations by separating reaction product(s) of single nucleotide primer extension by size and by detectable moiety.

7. The method of claim 6, wherein said detectable moiety is fluorescently labeled.

8. The method of claim 5, wherein said single nucleotide extension primer comprises a labeled ddNTP.

9. The method of claim 8, wherein said labeled ddNTP is fluorescently labeled.

10. The method of claim 1, wherein 2 or more fragments are amplified.

11. The method of claim 1, wherein 3 or more fragments are amplified.

12. The method of claim 1, wherein 4 or more fragments are amplified.

13. The method of claim 1, wherein at least 5 fragments are amplified.

14. The method of claim 1, wherein two or more fragments are amplified in the same vessel.

15. The method of claim 14, wherein said amplifying is accomplished with a multiplex polymerase chain reaction (PCR).

16. A method for determining the presence or absence of one or more mutations in the CYP2A6 gene from nucleic acids in a sample said method comprising:
(a) amplifying a fragment of the CYP2A6 gene, containing the site of the mutation, using at least one or more oligonucleotides specific to said fragment to create an amplified fragment;
(b) performing single nucleotide primer extension to detect the identity of the nucleotide added to the extension primer, wherein the identity of the nucleotide indicates the presence or absence of said one or more mutations in the CYP2A6 gene;
wherein at least one oligonucleotide suitable for amplifying said fragment comprises a sequence selected from the group consisting of: SEQ ID NOs: 21-30 and complements thereof.

17. The method of claim 16, wherein said single nucleotide primer extension comprises extension primers selected from the group consisting of: SEQ ID NOs: 41-59.

18. A method for detecting gene deletion, duplication, and/or rearrangement in the CYP2A6 gene from nucleic acids in a sample said method comprising:
(a) using at least one oligonucleotide capable of hybridizing to one or more of the sequences of SEQ ID NOs: 1-4, 7-10 or complements thereof,
(b) amplifying with said oligonucleotide at least one fragment of the CYP2A6 gene, containing the suspected gene deletion, duplication, and/or rearrangement, and
(c) detecting the deletion, duplication, and/or rearrangement in the CYP2A6 gene using dosage analysis,
wherein a substantial decrease or increase in the amount of detectable fragment observed indicates a deletion, duplication, and/or rearrangement of the CYP2A6 gene,
wherein said oligonucleotide is capable of specifically amplifying the CYP2A6 gene but not the CYP2A7 gene or pseudogenes of CYP2A6 and CYP2A7.

19. The method of claim 18, wherein at least one oligonucleotide suitable for amplifying said fragment comprises a sequence selected from the group consisting of: SEQ ID NOs: 22, 24, 28, 29, 37, 38, 39, 40 and complements thereof.

20. The method of claim 18, further comprising at least one or more oligonucleotides suitable for amplifying at least one internal control fragment that does not correspond to the CYP2A6 gene.

21. The method of claim 20, wherein said one or more oligonucleotides is selected from the group consisting of sequences of SEQ ID NOs: 31-36 and complements thereof.

22. The method of claim 20, wherein at least three internal control fragments are amplified.

23. The method of claim 18, wherein said one or more oligonucleotides is detectably labeled.

24. The method of claim 23, wherein said one or more oligonucleotides is fluorescently labeled.

25. The method of claim 18, wherein 2 or more fragments are amplified.

26. The method of claim 18, wherein 3 or more fragments are amplified.

27. The method of claim 18, wherein at least 4 fragments are amplified.

28. The method of claim 18, wherein two or more fragments are amplified in the same vessel.

29. The method of claim 18, wherein said amplifying is accomplished with a multiplex polymerase chain reaction (PCR).
